SEQUENCE LISTING (1 sequence)

SEQ ID NO: 1 - protein, 406 aa, Homo sapiens [sequence omitted]

(19) United States
(12) Patent Application Publication
(10) Pub. No.: US 2009/0011991 A1
(43) Pub. Date: Jan. 8, 2009

(54) Novel Curcuminoid-Factor VIIA Constructs as Suppressors of Tumor Growth and Angiogenesis

(75) Inventors: Mamoru Shoji, Conyers, GA (US); James Synder, Atlanta, GA (US); Dennis Liotta, Atlanta, GA (US); Aiming Sun, Atlanta, GA (US)

Correspondence Address: PATREA L. PABST;PABST PATENT GROUP LLP, 400 COLONY SQUARE, SUITE 1200, 1201 PEACHTREE STREET, ATLANTA, GA, 30361, US

(73) Assignee: Emory University

(21) Appl. No.: 12/035,995

(22) Filed: Feb. 22, 2008

Related U.S. Application Data
(63) Continuation-in-part of application No. 10/383,898, filed on Mar. 7, 2003.
(60) Provisional application No. 60/362,762, filed on Mar. 8, 2002.
(60) Provisional application No. 60/403,794, filed on Aug. 14, 2002.

(51) Int. Cl. A61K 38/36; A61P 35/00

(52) U.S. Cl. 514/12

(57) ABSTRACT

The fluorinated curcuminoid (3,5-bis-(2-fluorobenzylidene)-piperidin-4-one-acetate is about ten times more effective at arresting the growth of tumor cells than cisplatin. Conjugates for delivering a cytotoxic compound, such as a curcuminoid, specifically to cancer cells and to the vascular endothelial cells that nourish solid tumors, and methods of making and using thereof are described herein. The conjugate contains a cytotoxic compound bound to a protein such as in factor VIIa that retains high affinity for the surface protein tissue factor. The cytotoxic compound is bound to the protein via a linker and a hydrolyzable bond. Upon complexation, the resulting heterodimer is endocytosed and the drug is subsequently liberated inside the target cell via proteolytic cleavage.

### CROSS-REFERENCE TO RELATED APPLICATIONS

This application is a continuation-in-part of U.S. Ser. No., 10/383,898 entitled “Novel Curcuminoid-factor VIIa Constructs as Suppressors of Tumor Growth and Angiogenesis” by Mamoru Shoji, James Snyder, Dennis Liotta, and Aiming Sun, filed on Mar. 7, 2003, which claims priority to U.S. Ser. No. 60/362,762, filed on Mar. 8, 2002 and U.S. Ser. No. 60/403,794, filed Aug. 14, 2002.

### GOVERNMENT SUPPORT

This invention was made, at least in part, with funding from the National Institutes of Health with grant 1 R21 CA82995-01A1 and Department of Defense, Department of U.S. Army grant DAMD17-00-1-0241. Accordingly, the United States Government has certain rights in this invention.

### FIELD OF THE INVENTION

The present invention relates to conjugates for selectively delivering a curcuminoid to a target cell, pharmaceutical compositions containing the conjugate, and methods of making and using thereof.

### BACKGROUND OF THE INVENTION

The association between malignant disease and the hypercoagulable state was documented more than 100 years ago. A critical role for tumor-derived vasoactive factors like vascular endothelial growth factor (VEGF) in the formation of the blood vessels that nourish tumors has been emphasized in more recent work. Cell-associated procoagulants like tissue factor (TF) have also been implicated in the pathogenesis of these events. “Tissue factor” is a transmembrane protein receptor specific for coagulation factor VII (and its activated form factor VIIa (fVIIa)), and is the primary regulator of blood coagulation. When bound to the extracellular domain of TF, fVIIa activates factor X (fX) via the extrinsic pathway. Alternatively, TF-VIIa indirectly activates fX via the activation of factor IX in the intrinsic blood clotting pathway. Independent of the potent procoagulant function, TF may act as a modulator of VEGF expression and as a cell signal transducer. These studies have provided important evidence for a dynamic interaction between host inflammatory cells, tumor cells and vascular endothelial cells (VECs). “Leaky blood vessels,” perfusion of tumors with fibrinogen and conversion of the fibrinogen to fibrin by cell-associated procoagulants in the local tumor microenvironment are some of the consequences. These events may occur at the blood vessel wall during hematogenous spread of tumors or within the extravascular space as primary tumors or metastasis grow. Fibrin may be generated by the expression of procoagulant activity, particularly tissue factor expressed on the surface of tumor cells, tumor-associated macrophages and tumor-associated VECs.

Increased tumor angiogenesis is associated with a poor prognosis in a variety of human tumors, including invasive breast cancer, early stage and node negative breast cancer, prostate carcinoma and adenocarcinoma of the lung. There is a statistically significant correlation between so-called tumor microvascular density and relapse-free survival. It has been shown that tumor cells secrete a number of angiogenic factors, including VEGF, interleukin-8 (IL-8) and basic fibroblast growth factor (bFGF), and endothelial cell proliferation is faster in tumors compared with normal tissues.

Tumor cells secrete factors that increase vessel permeability. Vascular permeability factor, or VEGF, purified originally from tumor cells has a molecular weight of 45 kDa and acts specifically on VECs to promote vascular permeability, endothelial cell growth and angiogenesis. VEGF induces expression of TF activity in VECs and monocytes and is chemotactic for monocytes, osteoblasts and VECs. VEGF promotes extravasation of plasma fibrinogen, which can be converted to fibrin by TF-dependent mechanisms. Fibrin deposition alters the tumor extracellular matrix to promote the migration of macrophages, fibroblasts and endothelial cells.

Overexpression of the TF gene in murine tumor cells leads to increased VEGF and decreased transcription of thrombospondin (TSP), an endogenous antiangiogenic factor. When grown in immunodeficient mice, the TF-producing cells stimulate angiogenesis by approximately 2-fold, whereas low TF producers inhibit angiogenesis. This effect of TF is independent of its clot-promoting activated procoagulation activity. Human melanoma cells, transfected to hyperexpress TF, demonstrate greater metastatic potential than those with low TF expression. This pro-metastatic effect of TF requires the procoagulant function of the extracellular domain of TF and its cytoplasmic domain. Tissue Factor, therefore, regulates angiogenic properties of tumor cells by regulating the production of growth regulatory molecules that can act on VECs. There is also a critical role for TF expression in blood vessel development in both mice and human embryos. TF appears to have the dual function of regulating angiogenesis and vasculogenesis.

Malignant human breast cancers and melanomas express high levels of TF and VEGF. TF is also expressed on the surface of vascular endothelial cells (VECs) within the tumor micro environment of invasive breast cancer and adenocarcinoma of the lung. There is a strong relationship between the synthesis of TF and VEGF levels in human breast cancer cell lines and in human melanoma cell lines, and there is co-localization of TF- and VEGF-specific mRNAs.

The signal for VEGF synthesis in cancer cells is mediated via serine residues of the TF cytoplasmic tail which contains two serine residues that can be substrates for protein kinase C. Expression of TF and VEGF in cancer cells is further enhanced under hypoxic condition, and TF may function as a growth factor receptor. Factor VIIa may induce cell signaling via PKC-dependent phosphorylation, mitogen-activated protein kinase (MAPK) pathways and subsequently, via the transcription factors NF-.kappa.B and AP-1.

Curcumin, a yellow-colored spice used in curry and a product of turmeric, inhibits tumor necrosis factor- and phorbol ester-induced TF synthesis in VECs by blocking the transcription factors NF-.kappa.B, AP-1 and Egr-1. Curcumin can also inhibit TF and VEGF synthesis of human melanoma cell lines and prostate cancer cell lines, as well as bFGF-induced angiogenesis.

There exists a need for conjugates and methods for delivering cytotoxic compounds, such curcumin and curcumin derivatives (curcuminoids) to a specific target, e.g., TF, which is aberrantly expressed on tumor cells and vascular endothelial cells in the tumor micro-environment.

Therefore, it is an object of the invention to provide conjugates, pharmaceutical composition containing the conjugates, and methods of making and using thereof for delivering cytotoxic compounds, such curcumin and curcumin derivatives (curcuminoids) to a specific target, e.g., TF, which is aberrantly expressed on tumor cells and vascular endothelial cells in the tumor micro-environment.

### SUMMARY OF THE INVENTION

Conjugates containing one or more cytotoxic compounds such as synthetic antitumor and anti-angiogenesis curcumin analogs (curcuminoids) linked to a protein delivery vehicle that can deliver the cytotoxic compound to a target cell (e.g., cancer cells and vascular endothelial cells having surface-bound tissue factor) and methods of making and using the conjugates are described herein. The cytotoxic compound maybe covalently linked to a tether. The tether is covalently linked to a linker, for example, the N-terminal amino acid of a peptidyl linker such as phenylalanine-phenylalanine-arginine, the C-terminal amino acid of which comprises a methylketone. The methylketone group forms a covalent bond with an amino acid side group of factor VIIa (fVIIa) that does not prevent the conjugated construct from selectively binding to tissue factor expressed on a cell membrane. Preferably, the curcuminoid-tether-linker will be bound to an amino acid of the serine protease domain of the fVIIa, thereby blocking the procoagulating activity of the therapeutic composition.

The compositions may increase the efficacy of the cytotoxic agents and decrease their side effects by delivering the agents to specific target cells. One of the curcumin analogs, EF24, was about 10 times more potent than cisplatin, which is a well-known anticancer agent currently in clinical use. The conjugate EF24-FFRck-fVIIa construct described herein kills cancer cell lines and vascular endothelial cells, such as HUVECs, that express tissue factor on the cell surface. The conjugate does not kill normal cells that do not express tissue factor. EF24-FFRck that is not coupled to fVIIa does not kill either cancer cells or normal cells regardless of the presence or absence of tissue factor expression on the cell surface because it cannot bind to any cells. Unconjugated EF24 alone indiscriminatingly kills normal cells, as well as cancer cells, irrespective of the level of tissue factor expression on the cell surface.

The conjugates are particularly useful for delivering a drug to the blood vessels that feed cancer cells, thereby interrupting the supply of nutrients and oxygen and starving cancer cells. The methods are also useful for overcoming shortcomings of current cancer gene therapies that are unable to deliver drugs or genes intravenously because most cancers and their metastatic foci are inaccessible by a direct injection.

The conjugates described herein are able to deliver therapeutic agents to cancer cells, vascular endothelial cells in a tumor and metastatic foci anywhere in the body intravenously, intraperitoneally, subcutaneously, and intra-tumoraly, providing the target cells express surface bound tissue factor. The analogs are also coupled to fVIIa so as to inactivate the active site of fVIIa so that besides acting as anticancer agents the curcminoid-conjugated inactivated fVIIa may also inhibit blood clotting by competing with native fVIIa. This will be therapeutic advantage for cancer patients since many such patients experience blood clotting problems due to cancer cells that express tissue factor escaping into the circulation and triggering blood coagulation.

The conjugates are useful for treating any disease that requires targeted delivery of antiangiogenesis therapy including, but not limited to, reocclusion of the coronary artery. Restenosis occurs in 50% of angioplasty cases leading to myocardial infarction or angina pectoris. In angioplasty, the inner most layer of a treated blood vessel (vascular endothelial cells) is denuded. Tissue factor is then expressed on the exposed smooth muscle layer which proliferates and often re-obstructs the coronary artery. The conjugates therefore, are useful for delivering a drug specifically to the vascular smooth muscle cells that express tissue factor so as to inhibit the cell proliferation.

Other pathological conditions that may be regulated using the conjugates include, but are not limited to, diabetic retinopathy that also involves the uncontrollable growth of blood vessels, expressing tissue factor, in the retina and leads to blindness in diabetic patients. Brain infarction results from blood clots triggered by atherosclerosis and vasculitis where tissue factor is likely to be expressed. Blood vessels of early lesions of rheumatoid arthritis also express tissue factor.

In one embodiment, pharmaceutically acceptable compositions are prepared containing a therapeutically-effective amount of a cytotoxic composition-protein conjugate together with one or more pharmaceutically acceptable carriers (additives) and/or diluents for administering to an animal or human patient. The preferred route of administration is intravascular injection so that the effective dose of the curcuminoid can be delivered to a tumor via the vascular system. The dose may be delivered by subcutaneous injection, intraperitoneal injection, direct injection into the tumor or a proximal blood vessel feeding the tumor for reducing dilution of the effective therapeutic composition, and to achieve more rapid application of the composition to the tissue factor-bearing target tumor and/or vascular cells. The affinity of the fVIIa carrier polypeptide for tissue factor will localize the effective dose of the therapeutic composition for selectively targeting proliferating tumor and endothelial cells contributing to neovascularization of a tumor and to prevent metastasis of the tumor cells themselves.

### BRIEF DESCRIPTION OF THE DRAWINGS

FIG. 1 is the amino acid sequence (SEQ ID NO:1) of factor VII (fVIIa). Letters in bold indicate the cleavage point for conversion of the single-chain fVIIa to two-chain fVIIa, and the His193 that receives a covalently bonded arginyl-chloromethyl ketone of a peptidyl linker.

FIG. 2 is a graph showing the mean growth inhibitory concentrations (μM) of various curcuminoids when added to cultures of immortalized endothelial cells.

FIG. 3 is a graph showing the relative binding (measured as a percent) of EF24-FFRck-fVIIa conjugate, fluorescein-FPRck-fVIIa conjugate, and fVIIa to tissue factor (TF) as a function of concentration (picomoles) of the three materials.

FIGS. 4A-E are graphs showing tissue factor (TF)-dependent cytotoxic activity (expressed as percent cell viability) as a function of the concentration (μm) of EF24-FFRck-fVIIa conjugate, free EF24, and EF 24-FFRck. Percent cell viability was measured in human breast cancer cells MDA-MB-231 (FIG. 4A), normal human breast luminal ductal cell line MCF-10 (FIG. 4B), and normal HUVECs (FIGS. 4C and 4D). FIG. 4E is a graph showing the absence of cytotoxicity of the carrier, FFRck-VIIa against MDA-MB-231, PRMI, and DU 145 cell lines.

FIG. 5 is a graph showing the mean growth inhibitory concentrations (μM) of Curcumin, EF24, and cisplatin when tested against a panel of cultured tumor cells in the NCI screening system

FIG. 6 is a graph showing the mean growth inhibitory concentrations (μM) of various curcuminoids when added to cultures breast cancer cells.

### DETAILED DESCRIPTION OF THE INVENTION

### I. Definitions

As used herein, the singular forms “a,” “an” and “the” include plural references unless the content clearly dictates otherwise. Thus, for example, reference to “a carrier” includes a mixture of two or more carriers.

As used herein, the terms “polypeptide” and “protein” refer to a polymer of amino acids of three or more amino acids in a serial array, linked through peptide bonds. The term “polypeptide” includes proteins, protein fragments, protein analogues, oligopeptides and the like. The term “polypeptides” also contemplates polypeptides as defined above that are encoded by nucleic acids, produced through recombinant technology, isolated from an appropriate source, or are synthesized. The term “polypeptide” further contemplates polypeptides as defined above that include chemically modified amino acids or amino acids covalently or noncovalently linked to labeling ligands.

The term “truncated”, as used herein, refers to a polypeptide or protein that has less amino acids than a parent polypeptide or protein. It is contemplated that the difference in the amino acid sequence may be at one or both of the termini of an amino acid sequence or due to amino acids deleted from the interior of the sequence when compared to the parent amino acid sequence.

The term “linker”, as used herein, refers a molecule capable of covalently connecting a cytotoxic compound to an amino acid side chain of a protein. The term “linker” may be a non-peptidyl linker or a peptidyl linker. The linker may optionally have covalently bonded thereto a tether, as defined below, for covalently linking a cytotoxic compound to the linker. The term “peptidyl linker” as used herein refers to a peptide comprising at least two amino acids and which can be coupled to an amino acid side-chain of a protein. The linker may have a reactive group at the carboxyl terminus such as, but not limited to, a chloromethylketone. The peptide of the peptidyl linker may be cleavable by proteolytic enzymes found within a cell.

The term “tether”, as used herein, refers to a molecule that can form a hydrolysable bond such as, but not limited to, a carbamate, an amide, an ester, a carbonate or a sulfonate bond with a cytotoxic compound such as, but not limited to, a curcuminoid, and which can also be covalently bonded to a linker such as, but not limited to, the N-terminus of a linker, including a peptidyl linker, thereby connecting the cytotoxic compound to the linker. Suitable tethers for use in the present invention include, but are not limited to, a dicarboxylic acid, a disulfonic acid, an omega-amino carboxylic acid, an omega-amino sulfonic acid, an omega-amino carboxysulfonic acid, or a derivative thereof, wherein the tether may comprise 2-6 carbons in any arrangement such as a linear, branched or cyclic carbon arrangement, and wherein the tether is capable of forming a hydrolysable bond.

The term “cytotoxic compound”, as used herein, refers to a compound that, when delivered to a cell, either to the interior of a target cell or to the cell surface, is capable of killing the cell or otherwise inhibiting the proliferation of the target cell. The cytotoxic compound can be any such molecule that can form an amide or ester bond or otherwise be covalently bonded to a tether or a peptidyl linker and thereby connected to a protein that can selectively bind to a surface marker of a cell.

The terms “cell surface antigen” and “cell surface marker”, as used herein, may be any antigenic structure on the surface of a cell. The cell surface antigen may be, but is not limited to, a tumor associated antigen, a growth factor receptor, a viral-encoded surface-expressed antigen, an antigen encoded by an oncogene product, a surface epitope, a membrane protein which mediates a classical or atypical multi-drug resistance, an antigen which mediates a tumorigenic phenotype, an antigen which mediates a metastatic phenotype, an antigen which suppresses a tumorigenic phenotype, an antigen which suppresses a metastatic phenotype, an antigen which is recognized by a specific immunological effector cell such as a T-cell, and an antigen that is recognized by a non-specific immunological effector cell such as a macrophage cell or a natural killer cell. Examples of “cell surface antigens” within the scope of the present invention include, but are not limited to, CD5, CD30, CD34, CD45RO, CDw65, CD90 (Thy-1) antigen, CD117, CD38, and HLA-DR, AC133 defining a subset of CD34.sup.+ cells, CD 19, CD20, CD24, CD10, CD13, CD33 and HLA-DR. Also contemplated to be within the scope of the present invention are cell surface molecules, including carbohydrates, proteins, lipoproteins or any other molecules or combinations thereof, that may be detected by selectively binding to a ligand or labeled molecule by methods such as, but not limited to, flow cytometry, FRIM, fluorescence microscopy and immunohistochemistry.

The term “tissue factor”, as used herein, refers to a transmembrane protein receptor for coagulation factor VII (and the activated form factor VIIa (fVIIa)), and is the primary regulator of blood coagulation.

The term “fVII”, as used herein, refers to “single chain” coagulation factor VII that may have the amino acid sequence SEQ ID NO: 1, or a trucncated or modified form thereof.

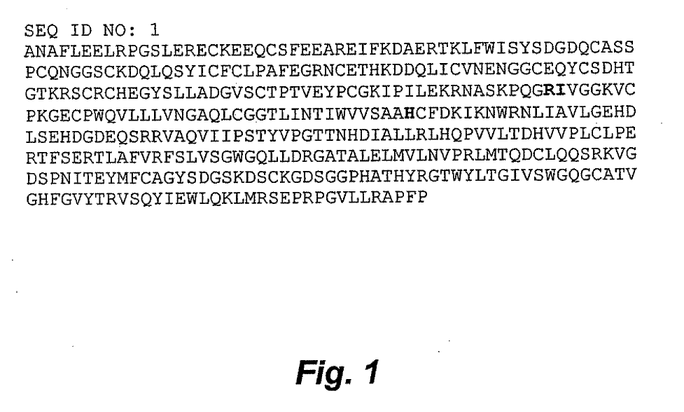

The term “factor VIIa”, or “fVIIa”, as used herein, refers to “two chain” activated coagulation factor VII cleaved by specific cleavage at the Arg152-Ile153 peptide bond. The uncleaved factor VII has the contiguous sequence as illustrated in FIG. 1. Factor VIIa, may be purified from blood or produced by recombinant means. It is evident that the practice of the methods described herein is independent of how the purified factor VIIa is derived and, therefore, the present invention is contemplated to cover use of any factor VIIa preparation suitable for use herein. It is anticipated that the covalent bonding of the linker to the polypeptide may be to the uncleaved factor VII which is subsequently cleaved between the 152-153 amino acid positions, or to the cleaved fvIIa.

The term “angiogenesis inhibitor”, as used herein, refers to a compound or composition that, when administered as an effective dose to an animal or human, will inhibit or reduce the proliferation of vascular endothelial cells, thereby reducing the formation of neovascular capillaries.

Angiogenesis inhibitors may be divided into at least two classes. The first class, direct angiogenesis inhibitors, includes those agents which are relatively specific for endothelial cells and have little effect on tumor cells. Examples of these include soluble vascular endothelial growth factor (VEGF) receptor antagonists and angiostatin.

Indirect inhibitors may not have direct effects on endothelial cells but may down-regulate the production of an angiogenesis stimulator, such as VEGF. (Arbiser et al., Molec. Med. 4:376-383 (1998)). VEGF has been shown to be up-regulated during chemically induced skin carcinogenesis; this is likely due to activation of oncogenes such as H-ras. (Arbiser et al., Proc. Natl. Acad. Sci. U.S.A. 94:861-866 (1997)); (Larcher et al., Cancer Res. 56:5391-5396 (1996)); (Kohl et al., Nature Med. 1:792-797 (1995)). Examples of indirect inhibitors of angiogenesis include inhibitors of ras-mediated signal transduction, such as farnesyltransferase inhibitors.

Direct inhibition of endothelial cell proliferation can be assayed in cell culture systems, in which the effects of specific factors which control the complex process of angiogenesis can be studied. Effects discovered in such in vitro systems can then be studied in in vivo systems as described, for example, by Kenyon et al., Invest. Opthalmol. 37:1625-1632 (1996).

The term “curcumin (diferuloylmethane)” and certain of its analogs, together termed “curcuminoids,” as used herein, refers to well known natural product, recognized as safe for ingestion by and administration to mammals including humans. (Bille et al., Food Chem. Toxicol. 23:967-971 (1985)). The term “curcuminoid” as used herein also refers to synthetic curcumin derivatives such as, but not limited to those disclosed in PCT Application Serial No. WO 01/40188 incorporated herein by reference in its entirety. Curcumin is a yellow pigment found in the rhizome of Curcuma longa, the source of the spice turmeric. Turmeric has been a major component of the diet of the Indian subcontinent for several hundred years, and the average daily consumption of curcumin has been found to range up to 0.6 grams for some individuals, without reported adverse effects. Food-grade curcumin consists of the three curcuminoids in the relative amounts: 77% curcumin, 17% demethoxycurcumin, and 3% bisdemethoxycurcumin.

The fully saturated derivative tetrahydrocurcumin is also included in the term curcuminoid. Curcumin can be obtained from many sources, including for example Sigma-Aldrich, Inc. The curcumin analogs demethoxycurcumin, bisdemethoxycurcumin and tetrahydrocurcumin can also be obtained from many sources, or readily prepared from curcumin by those skilled in the art.

Curcumin has been used in indigenous Indian medicine for several hundred years, as a topical agent for sprains and inflammatory conditions, in addition to oral use to promote health and treat digestive and other disorders. Absorption of ingested or orally administered curcumin is known to be limited, and absorbed curcumin is rapidly metabolized. (Govindarajan, CRC Critical Rev. Food Sci Nutr. 12:199-301 (1980); Rao et al., Indian J. Med. Res. 75:574-578 (1982)).

Numerous effects of the ingestion or oral administration of the curcuminoids have been reported, based on controlled research, population studies, case reports and anecdotal information. Evidence of chemopreventive activity of curcumin administered orally has led to clinical trials sponsored by the National Cancer Institute, regarding prevention of cancer. (Kelloff et al., J. Cell. Biochem. Suppl. 26: 1-28 (1996)). Oral administration of curcumin to mice treated with skin and colon chemical carcinogens has been shown to result in a decreased incidence and size of induced tumors compared with control mice. (Huang, et al., Cancer Res. 54:5841-5847 (1994); Huang et al., Carcinogenesis 16:2493-2497 (1995); Huang et al., Cancer Lett. 64:117-121; Rao et al., Cancer Res. 55:259-266 (1995); Conney et al., Adv Enzyme Regul. 31:385-396 (1991)).

Huang, et al. found that the oral administration of three curcuminoid compounds curcumin, demethoxycurcumin and bisdemethoxycurcumin were able to inhibit phorbol ester-stimulated induction of ornithine decarboxylase and promotion of mouse skin initiated with 7,12-dimethylbenzanthracene (DMBA). These compounds also inhibited phorbol ester-mediated transformation of JB6 cells. The saturated derivative tetrahydrocurcumin was less active than the unsaturated analogs in these assays. Huang et al., Carcinogenesis 16:2493-2497 (1995).

The mechanism or mechanisms of curcumin's chemopreventive activities were not previously understood, although it was recognized as an antioxidant and was known to exhibit antimutagenic activity in the Ames Salmonella test and to produce biochemical effects similar to those of the polyphenols, chemopreventive agents found in green tea. Stoner, J. Cell. Biochem. Suppl. 22:169-180 (1995). Curcumin has been demonstrated to inhibit several signal transduction pathways, including those involving protein kinase, the transcription factor NF-.kappa.B, phospholipase A2 bioactivity, arachidonic acid metabolism, antioxidant activity, and epidermal growth factor (EGF) receptor autophosphorylation. Lu et al., Carcinogenesis 15:2363-2370 (1994); Singh et al., J. Biol. Chem. 270:24995-25000 (1995); Huang et al., Proc. Natl. Acad. Sci. U.S.A. 88:5292-5296 (1991); Korutla et al., Carcinogenesis 16:1741-1745 (1995); Rao et al., Carcinogenesis 14:2219-2225 (1993).

Because of the complexity of the factors that regulate or effect angiogenesis, and their specific variation between tissues and according to circumstances, the response to a specific agent may be different or opposite, in different tissues, under different physiological or pathological conditions and between in vitro and in vivo conditions. For example, U.S. Pat. No. 5,401,504 to Das et al., describes the oral or topical administration of turmeric to animals and humans and alleges that such administration promotes wound healing, and further postulates that it acts in part through stimulation of angiogenesis, although this postulate was not experimentally verified. Administration of curcumin has been reported to inhibit smooth muscle cell proliferation in vitro. Huang et al., European J. Pharmac. 221:381-384 (1992). U.S. Pat. No. 5,891,924 to Aggarwal alleges that oral administration of curcumin to animals inhibits activation of the transcription factor NF-.kappa.B, and claims its use in pathophysiological states, particularly specific conditions involving the immune system. Several biochemical actions of curcumin were studied in detail, but no single action was reported to be responsible for these effects of curcumin. Singh et al., Cancer Lett. 107:109-115 (1996) reported that curcumin inhibits in vitro proliferation of human umbilical vein endothelial cells (HUVEC) and suggested that it might have anti-angiogenic activity. However, this inhibition was independent of basic fibroblast growth factor stimulation of the proliferation of endothelial cells, and in vivo studies were not reported. Inhibition by curcumin of HUVEC growth and formation of tube structures on Matrigel, in a model of capillary formation, has been ascribed to modulation of metalloproteinases of the HUVEC. (Thaloor et al., Cell Growth Differ. 9:305-312 (1998)).

The term “prodrug”, as used herein, refers to compounds which, under physiological conditions, may be converted into a pharmaceutically active curcuminoid of the present invention. A common method for making a prodrug is to select moieties which are hydrolyzed under physiological conditions to provide the desired biologically active drug. In other embodiments, the prodrug may be converted by an enzymatic activity of the recipient animal or cell.

The terms “methylketone” and “chloromethylketone”, as used herein, refer to the carboxy terminus reactive moiety that may form the covalent bond between a peptide linker and an amino acid side chain of a recipient polypeptide. During the linkage reaction, the chloro group is removed. Thus, the unlinked peptidyl linker will have a chloromethylketone moiety and the covalently attached peptide will have a methylketone moiety without a halogen atom thereon.

The term “aliphatic group”, as used herein, refers to a straight-chain, branched-chain, or cyclic aliphatic hydrocarbon group and includes saturated and unsaturated aliphatic groups, such as an alkyl group, an alkenyl group, and an alkynyl group.

The term “alkyl”, as used herein, refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In preferred embodiments, a straight chain or branched chain alkyl has 30 or fewer carbon atoms in its backbone (e.g., C₁-C₃₀ for straight chain, C₃-C₃₀ for branched chain), and more preferably 20 or fewer. Likewise, preferred cycloalkyls have from 3-10 carbon atoms in their ring structure, and more preferably have 5, 6 or 7 carbons in the ring structure. Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, alkylaminos, carbonyl-substituted alkyls, —CF₃, —CN, and the like.

The term “aralkyl”, as used herein, refers to an alkyl group substituted with an aryl group (e.g., an aromatic or heteroaromatic group).

The terms “alkenyl” and “alkynyl”, as used herein, refer to unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

The term “aryl”, as used herein, includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The term “aryl” refers to both substituted and unsubstituted aromatic rings. The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocycles.

The terms ortho, meta and para apply to 1,2-, 1,3- and 1,4-disubstituted benzenes, respectively. For example, the names 1,2-dimethylbenzene and ortho-dimethylbenzene are synonymous.

The terms “heterocyclyl” or “heterocycle”, as used herein, refer to 4- to 10-membered ring structures, more preferably 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, quinoline, pteridine, carbazole, carboline, phenanthridine, acridine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group”, as used herein, refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle”, as used herein, refers to an aromatic or non-aromatic ring in which each atom of the ring is carbon.

The phrase “fused ring” is art recognized and refers to a cyclic moiety which can comprise from 4 to 8 atoms in its ring structure, and can also be substituted or unsubstituted, (e.g., cycloalkyl, a cycloalkenyl, an aryl, or a heterocyclic ring) that shares a pair of carbon atoms with another ring. To illustrate, the fused ring system can be a isobenzofuran and a isobenzofuranone.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

The terms “amine” and “amino” are art-recognized and refer to both unsubstituted and substituted amines. The term “alkylamine” as used herein means an amine group, as defined above, having a substituted or unsubstituted alkyl attached thereto.

The term “amido” is art recognized as an amino-substituted carbonyl.

The term “alkylthio” refers to an alkyl group, as defined above, having a sulfur radical attached thereto. In preferred embodiments, the “alkylthio” moiety is represented by one of —S-alkyl, —S-alkenyl, —S-alkynyl, and —S—(CH₂)ₘ. Representative alkylthio groups include methylthio, ethylthio, and the like.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —O—(CH₂)ₘ.

Analogous substitutions can be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

As used herein, the definition of each expression, e.g. alkyl, m, n, etc., when it occurs more than once in any structure, is intended to be independent of its definition elsewhere in the same structure.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group or other stereogenic centers. All such isomers, as well as mixtures thereof, are intended to be included in this invention. Likewise certain compounds can display overall molecular asymmetry without stereogenic centers leading to sterioisomers

If, for instance, a particular enantiomer of a compound of the present invention is desired, it may be prepared by asymmetric synthesis, or by derivitization with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts can be formed with an appropriate optically-active acid or base, followed by resolution of the diastereomers thus formed by fractional crystallization or chromatographic means well known in the art.

The term “antibody”, as used herein, refers to polyclonal and monoclonal antibodies and fragments thereof, and immunologic binding equivalents thereof that are capable of selectively binding to a region of tissue factor. The term “antibody” refers to a homogeneous molecular entity, or a mixture such as a polyclonal serum product made up of a plurality of different molecular entities, and may further comprise any modified or derivatised variant thereof that retains the ability to specifically bind an epitope. A monoclonal antibody is capable of selectively binding to a target antigen or epitope.

The phrase “therapeutically-effective amount”, as used herein, means that amount of a compound, material, or composition comprising a curcuminoid linked to a polypeptide such as, but not limited to, fVIIa by means of a tether and a linker according to the present invention, and which is effective for producing some desired therapeutic effect against cancer or other pathological comprising neovascularization.

The phrase “pharmaceutically acceptable”, as used herein, refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier”, as used herein, refers to a pharmaceutically-acceptable material, composition or vehicle, such as a liquid or solid filler, diluent, excipient, solvent or an encapsulating material such as liposomes, polyethylene glycol (PEG), PEGylated liposomes, nonoparticles and the like, involved in carrying or transporting the subject curcuminoid-FFRck-fVIIa agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The phrases “parenteral administration” and “administered parenterally”, as used herein, means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally”, as used herein, mean the administration of a compound, drug or other material other than directly into the central nervous system, such that it enters the patient's system and, thus, is subject to metabolism and other like processes, for example, subcutaneous administration.

Abbreviations: Tissue factor, TF; vascular endothelial cell, VEC; vascular endothelial cell growth factor, VEGF; phenylalanine-phenylalanine-arginyl-(chloro) methylketone, FFR-ck; factor VII(a), fVII(a); active site-inactivated fVIIa, fVIIa-i; tissue factor pathway inhibitor, TFPI.

### II. Compositions

A. Curcuminoid-Factor VIIa Conjugates

Conjugates containing a cytotoxic compound covalently bound to a protein capable of selectively binding to a cell surface maker, and at least one linker for bonding the compound to the protein are described herein. The compositions may also contain a tether molecule that alone or in conjunction with the linker may serve to bond the cytotoxic compound to the protein.

The polypeptide is capable of selectively binding to a region (preferably an extracellular region) of a cell surface marker that is an integral component of a cell surface membrane of a target cell such as a vascular endothelial cell. In the various compositions of the present invention, the curcuminoid may be covalently bonded to a tether, which preferably is selected from, but not limited to, a dicarboxylic acid or caproyl moiety. An exemplary tether is succinate that may be bonded to a curcuminoid by the addition of succinic anhydride, as described in Example 2, below. It is, however, also considered to be within the scope of the present invention for any suitable therapeutic compound including, but not limited to curcumin analogs, anticancer drugs or cardiovascular agents, to be conjugated to a linker and a protein by the methods of the present invention, thereby reducing a required effective dose of the agent or drug and to reduce undesirable side effects, by directing the conjugated therapeutic agent to a selected target cell having a surface-exposed marker such as factor.

1. Proteins and Peptides

A suitable polypeptide for use in the conjugates may be any polypeptide that can selectively bind to a cell surface marker such as, for example, an extracellular region of surface bound tissue factor and which, when so bound, may then be internalized by the targeted cell. Suitable polypeptides include, but are not limited to factor VII or factor VIIa (fVIIa), tissue factor pathway inhibitor (TFPI) or an antibody capable of specifically binding to tissue factor and the like. The protein or peptide may also be a component polypeptide of fVIIa derived from the amino acid sequence SEQ ID NO: 1 shown in FIG. 1, wherein before conjugation to the linker, the polypeptide may be the uncleaved SEQ ID NO: 1, or cleaved between amino acid positions 152-153 such that the component polypeptide receiving the linker may comprise the amino acid sequence between positions 1 and 152, 153-406 or derivatives thereof of SEQ ID NO: 1. If the linker is conjugated to the uncleaved amino acid sequence, it is contemplated that the polypeptide may then be cleaved to the fVIIa dipeptide.

In one embodiment, the peptide is fVIIa having at least 80% similarity to the amino acid sequence SEQ ID NO:1, as shown in FIG. 1, cleaved between amino acid positions 152 and 153 or truncated derivatives or variants thereof. The fVIIa can be derived from any species, including human fVII. The fVIIa polypeptide may be truncated to include sequence variations by methods well known to those skilled in the art, including modification of cloned nucleic acid encoding all or part of SEQ ID NO:1, or by proteolytic cleavage of the fVIIa polypeptide, and the like. Any truncation or amino acid substitution will retain the ability of the modified fVIIa and or modified TFPI to selectively bind to tissue factor, be internalized by a target cell and capable of forming a covalent bond with a linker molecule, for example a linker having a chloromethylketone group thereon.

2. Linkers

The compositions may further contain a linker. In one embodiment, the linker is covalently bonded to an amino acid side chain within a serine protease active site of factor VIIa, thereby inactivating the serine protease active site. In a preferred embodiment, the linker is a peptidyl methylketone linker covalently bonded to the polypeptide, most preferably to the side chain of an amino acid within the catalytic triad of the serine protease domain of fVIIa. In the human and bovine factor VII proteins, the amino acids which form a catalytic “triad” are Ser344, Asp242, and His193, numbering indicating position within the sequence SEQ ID NO:1. The catalytic sites in factor VII from other mammalian species may be determined using presently available techniques including, among others, protein isolation and amino acid sequence analysis. Catalytic sites may also be determined by aligning a sequence with the sequence of other serine proteases, particularly chymotrypsin, whose active site has been previously determined by Sigler et al., J. Mol. Biol., 35:143-164 (1968), incorporated herein by reference, and therefrom determining from said alignment the analogous active site residues. Attachment of the peptidyl linker to this domain will inactivate the serine protease activity, thereby reducing the potential of the composition, when administered to an animal, to induce blood coagulation. In one preferred embodiment of the present invention, at least one linker is covalently bonded to the His 198 position of SEQ ID NO:1.

In one embodiment, suitable peptidyl linkers, before being bound to the polypeptide, have a carboxy-terminus chloromethylketone group that may react with a suitable amino acid side chain of the polypeptide, as described in Example 3, below. Preferably, but not necessarily, the carboxy terminal amino acid having the chloromethylketone group thereon is an arginine. Although any peptidyl chain sequence or length may be used in the compositions of the present invention, a suitable peptide is a tripeptide. Preferred peptidyl linkers include, but are not limited to, tyrosine-glycine-arginine-chloromethylketone (YGR-ck); phenylalanine-phenylalanine-arginine-chloromethylketone (FFR-ck), glutamine-glycine-arginine-chloromethylketone (QGR-ck), glutamate-glycine-arginine chloromethylketone (EGR-ck) and the like. A most preferred linker is FFR-ck. The stoichiometry of attachment of the curcuminoid EF24-tether-FFRck to fVIIa is given in Example 4, below.

It will be understood by those of skill in the art that upon covalently attaching the chloromethylketone to the recipient polypeptide, the chloro-moiety is displaced. Accordingly, the term “FFR-ck-VIIa”, for example, refers to FFR-methylketone tripeptidyl linker bonded to fVIIa and not having a chloro-atom attached thereto.

While not wishing to be bound by any one theory, a complex, formed from phenylalanyl-phenylalanyl-arginyl-ck-VIIa (FFR-ck-VIIa) and tissue factor (TF) expressed on the plasma membrane of cancer cells, may be internalized in a FFR-ck-VIIa concentration-dependent manner by ligand-receptor mediated endocytosis. The ligand-receptor complex is endocytosed into early and late endosomes and is delivered to lysosomal vesicles and degraded by lysosomal enzymes.

The peptide selected for use as a linker peptide in the compositions is also suitable for cleavage by an intracellular hydrolytic activity of the target cell enzyme. When so cleaved, after endocytotic internalization, the curcuminoid attached to the linker may be released from a polypeptide such as fVIIa. The released curcuminoid may then modulate a physiological function of the target cell.

3. Tethers

The conjugate can also contain a tether. “Tether” refers to a molecule that can form a hydrolysable bond such as, but not limited to, a carbamate, an amide, an ester, a carbonate or a sulfonate bond with a cytotoxic compound such as, but not limited to, a curcuminoid, and which can also be covalently bonded to a linker such as, but not limited to, the N-terminus of a linker, including a peptidyl linker, thereby connecting the cytotoxic compound to the linker. Suitable tethers for use in the present invention include, but are not limited to, a dicarboxylic acid, a disulfonic acid, an omega-amino carboxylic acid, an omega-amino sulfonic acid, an omega-amino carboxysulfonic acid, or a derivative thereof, wherein the tether may comprise 2-6 carbons in any arrangement such as a linear, branched or cyclic carbon arrangement, and wherein the tether is capable of forming a hydrolysable bond. In one embodiment, the linker is a tether.

In a preferred embodiment, the tether is derived from a dicarboxylic acid, such as succinate.

4. Cytotoxic Compounds

i. Curcumin Analogs

More than ninety curcumin analogs have been synthesized, as described in PCT application serial number WO 01/40188, which is incorporated herein by reference in its entirety. Several of these compounds suppress cancer cell VEGF production, but are not cytotoxic to either cancer cells or endothelial cells at concentrations where curcumin is otherwise cytotoxic.

In one embodiment, the curcumin analog has Formula II:

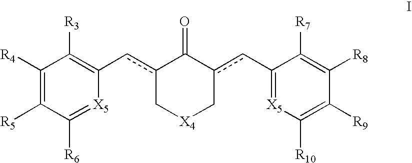

wherein X₄ is (CH₂)ₘ, O, S, SO, SO₂, CHNH₂, CHOH, CO, or NR₁₂, where R₁₂ is H, alkyl, substituted alkyl, acyl, alkoxycarbonyl, aminocarbonyl, alkylaminocarbonyl or dialkylaminocarbonyl; m is 1-7; each X₅ is independently N or C—R₁₁; and each R₃-R₁₁ are independently H, halogen, hydroxyl, alkoxy, CF₃, alkyl, substituted alkyl, alkenyl, alkynyl, cycloalkyl, substituted cycloalkyl, aryl, substituted aryl, alkaryl, arylalkyl, heteroaryl, substituted heteroaryl, heterocycle, substituted heterocycle, amino, alkylamino, dialkylamino, carboxylic acid, carboxylic ester, carboxamide, nitro, cyano, azide. alkylcarbonyl, acyl, or trialkylammonium; and the dashed lines indicate optional double bonds; with the proviso that when X₃ is (CH₂)ₘ, m is 2-6, and each X₅ is C—R₁₁, R₃-R₁₁ are not alkoxy, and when X₄ is NR₁₂ and each X₅ is N, R₃-R₁₀ are not alkoxy, alkyl, substituted alkyl, alkenyl, alkynyl, cycloalkyl, substituted cycloalkyl, aryl, substituted aryl, alkaryl, arylalkyl, heteroaryl, substituted heteroaryl, amino, alkylamino, dialkylamino, carboxylic acid, or alkylcarbonyl, and wherein the stereoisomeric configurations include enantiomers and diastereoisomers, and geometric (cis-trans) isomers.

Suitable analogs include, but are not limited to, those analogs wherein X₄ is selected from the group consisting of —NH and —NR₁₂, and R₃-R₁₀ may be selected from hydroxyl and —NHR₁₂.

A particularly suitable curcuminoid for use in the compositions of the present invention is 3,5-Bis-(2-fluorobenzylidene)-piperidin-4-one (EF24 having Formula II):

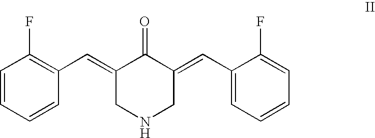

or a salt thereof.

It is contemplated that any curcuminoid such as, but not limited to, those curcuminoids disclosed in WO01/40188 may be used in the conjugates if capable of bonding to a carboxylic or polycaproyl tether by reactions such as described, for example, in Example 2, below. Methods for synthesizing the curcuminoids are described in detail in WO 01/40188.

The active region of the EF24 drug molecule (i.e. the series of double bonds between fluorine F, oxygen O, and F) is exposed and available in both the unconjugated and conjugated drugs. Either variation can be expected to exhibit cytotoxicity regardless of whether the drug is cleaved from the carrier inside the cells. Thus, the agent can be expected to show activity whether in the conjugated or free state. The drug has the potential to modulate the action of multiple targets by the mechanism of Michael addition by binding —SH groups of intracellular molecules. The existence of multiple targets, rather than a single target, may prove to be a decisive advantage, since it can reduce the occurrence of resistant cells, while diminishing toxicity.

B. Pharmaceutical Compositions Containing Cytotoxic Compound-Protein Conjugates

The conjugates described herein can be formulated with one or more pharmaceutically acceptable carriers and/or excipients for use as a therapeutic agent for the treatment of a pathological condition of an animal or human such as a cancer or a neovascular-based disease. The pharmaceutical compositions may be formulated for administration in solid or liquid form, including those adapted for oral administration or parenteral administration, for example, by subcutaneous, intramuscular or intravenous injection as, for example, a sterile solution or suspension. Conventional techniques for preparing pharmaceutical compositions which can be used according to the present invention are, for example, described in Remington's Pharmaceutical Sciences, 1985. The pharmaceutical compositions are intended for parenteral, topical or local administration for prophylactic and/or therapeutic treatment. Pharmaceutical compositions can be made mixing the conjugate preferably in purified form, with suitable adjuvants and/or a suitable carrier or diluent. Suitable physiological acceptable carriers or diluents include sterile water and saline. Suitable adjuvants, in this regard, include calcium, proteins (e.g. albumins), or other inert peptides (e.g. glycylglycine) or amino acids (e.g. glycine, or histidine) to stabilise the purified factor VIIa. Other physiological acceptable adjuvants are non-reducing sugars, cyclodextrins (cyclic carbohydrates derived from starch), polyalcohols (e.g. sorbitol, mannitol or glycerol), polysaccharides such as low molecular weight dextrins, detergents (e.g. polysorbate) and antioxidants (e.g. bisulfite and ascorbate). The adjuvants are generally present in a concentration of, but not limited to, from 0.001 to 4% w/v. The pharmaceutical preparation may also contain protease inhibitors, e.g. aprotinin, and preserving agents.

The preparations may be sterilized by, for example, filtration through a bacteria-retaining filter, by incorporating sterilizing agents into the compositions, by irradiating the compositions. They can also be manufactured in the form of sterile solid compositions which can be dissolved in sterile water, or some other sterile medium suitable for injection prior to or immediately before use.

Certain embodiments contain curcuminoids or derivatives thereof that may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of curcuminoids. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19).

In other cases, the compounds may contain one or more acidic functional groups and, thus, are capable of forming pharmaceutically-acceptable salts with pharmaceutically-acceptable bases. The salts can likewise be prepared in situ during the final isolation and purification of the curcuminoid containing composition of the present invention, or by separately reacting derivatives comprising carboxylic or sulfonic groups with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

1. Parenteral Formulations

The conjugates described herein can be formulated for parenteral administration. Parenteral administration allows the compositions to be rapidly transported to a selected target cell such as a cancer cell or neovascular endothelial cell. The formulations may conveniently be presented in unit dosage form and may be prepared by any methods well known in the art. The amount of active ingredient which can be combined with a carrier material to produce a single dosage form will vary depending upon the host being treated, the particular mode of administration, the age and weight of the host to be treated, as well as other factors. The attending physician can readily determine an effective amount based on these and other factors.

Pharmaceutical compositions for parenteral administration may contain one or more pharmaceutically-acceptable sterile isotonic aqueous or nonaqueous solutions, dispersions, suspensions or emulsions, or sterile powders which may be reconstituted into sterile injectable solutions or dispersions just prior to use. The formulations may also contain one or more pharmaceutically acceptable excipients including, but not limited to, antioxidants, buffers, bacteriostats, solutes, which render the formulation isotonic with the blood of the intended recipient, suspending or thickening agents, and combinations thereof.

Examples of suitable aqueous and nonaqueous carriers which may be employed include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and other antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as polyethylene glycol (PEG), aluminum monostearate and gelatin.

In some cases, in order to prolong the effect of a drug, it is desirable to slow the absorption of the drug from subcutaneous or intramuscular injection. This may be accomplished by coupling to PEG, the use of a liquid suspension of crystalline or amorphous material having poor water solubility. The rate of absorption of the drug then depends upon its rate of dissolution which, in turn, may depend upon size, form and amount of PEG, crystal size and crystalline form. Alternatively, delayed absorption of a parenterally-administered drug form is accomplished by dissolving or suspending the drug in an oil vehicle.

Injectable depot forms are made by forming microencapsuled matrices of the subject peptides or peptidomimetics in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The modified fVIIa molecules can also be formulated into liposome preparations for delivery or targeting to sites of injury. Liposome preparations are generally described in, e.g., U.S. Pat. No. 4,837,028, U.S. Pat. No. 4,501,728, and U.S. Pat. No. 4,975,282, incorporated herein by reference. The compositions may be sterilized by conventional, well known sterilization techniques. The resulting aqueous solutions may be packaged for use or filtered under aseptic conditions and lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, etc. The concentration of modified factor VII in these formulations can vary widely, i.e., from less than about 0.5%, usually at or at least about 1% to as much as 15 or 20% by weight and will be selected primarily by fluid volumes, viscosities, etc., in accordance with the particular mode of administration selected.

Thus, a desirable exemplary pharmaceutical composition for intravenous infusion could be made up to contain 0.05-5 mg/kg body weight (in rats) or 0.05-10 mg/kg human adult in 250 ml of sterile Ringer's solution, and 10 mg of modified factor VII. Actual methods for preparing parenterally administrable compounds will be known or apparent to those skilled in the art and are described in more detail in for example, Remington's Pharmaceutical Science, 16th ed., Mack Publishing Company, Easton, Pa. (1982), which is incorporated herein by reference.

### III. Methods of Making the Conjugates

The conjugates can be synthesized using a variety of methodologies known in the art. In on embodiment, the conjugate is prepared by (a) synthesizing a product containing a curcuminoid of Formula I having a tether covalently bonded thereto; (b) providing a peptidyl chloromethylketone linker; (c) bonding covalently the product of step (a) and the linker; and (d) covalently bonding the composition of step (c) to a polypeptide capable of selectively binding to tissue factor on the surface of a target cell. In a preferred embodiment, the curcuminoid is EF24. In another embodiment, the method includes the steps of synthesizing a product containing a cytotoxic compound, bonding covalently the product of step (a) and the linker, and covalently bonding at least one molecule of the composition of step (b) to a protein capable of selectively binding to a surface marker of a target cell.

A. Curcuminoid Functionalized with a Tether

In one embodiment step (a) includes reacting the curcuminoid with a tether selected from the group consisting of a dicarboxylic acid, a dicarboxylic anhydride, a disulfonic acid, an omega-amino carboxylic acid, an omega-amino sulfonic acid, an omega-amino carboxysulfonic acid, or a derivative thereof; wherein the tether contains 2-6 carbons, and wherein the tether is capable of forming a hydrolysable bond.

The structure of EF24 modified with a succinate tether is shown in Formula III.

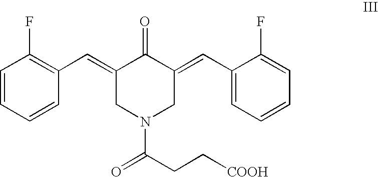

B. Curcuminoid Functionalized with a Tether and Linker

The curcuminoid having a tether covalent bound thereto can be further functionalized with a linker, such as a peptidyl linker. The preparation of an exemplary peptide linker is described below. The starting material of Formula IV:

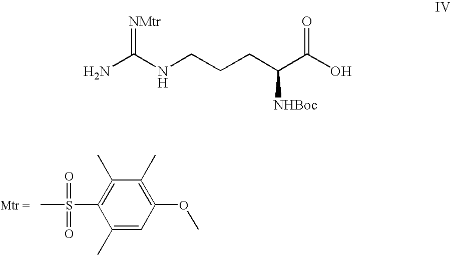

is reacted with isopropyl chloroformate and ethereal diazomethane to form the compound of Formula V:

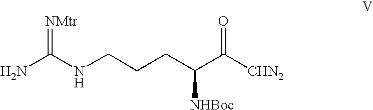

which is the reacted with hydrochloric acid in ethanol to produce the compound of Formula VI:

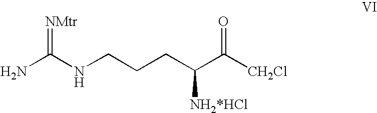

The compound of Formula VI is reacted with N-Boc-Phe-Phe-OH, isopropyl chloroformate, and a base to produce the compound of Formula VII:

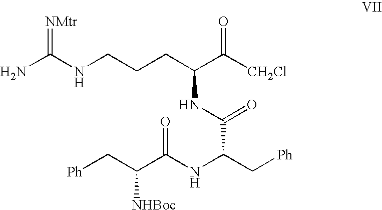

The compound of Formula VII is deprotected to produce the compound of Formula VIII:

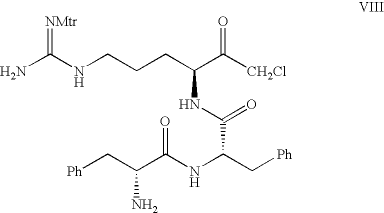

The compound of Formula VIII is reacted with the compound of Formula III to produce the compound of Formula IX

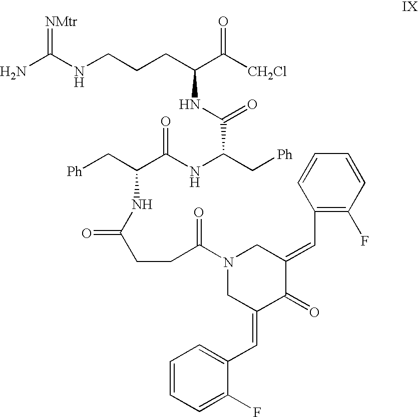

The Mtr protecting group can be removed by reacting the compound of Formula IX with trifluoroacetic acid and water. Finally, the compound of Formula IX is bound to a component polypeptide of Factor VIIa, preferably an amino acid of the serine protease active site of factor VIIa thereby inactivating the active site, via the chloromethylketone group. In one embodiment, the amino acid is His193 of SEQ ID NO: 1.

### III. Methods of Use

The conjugates described herein can be used to modulate a physiological function of a target cell by contacting a target cell having a surface marker thereon with a composition containing a cytotoxic compound-protein conjugate, wherein the composition selectively binds to the surface marker and is internalized, thereby releasing the cytotoxic compound from the protein; and modulating the physiological function of the target cells. In one embodiment the surface marker is tissue factor.

It has been reported that the angiogenic switch is turned on when microscopic tumors reach at a millimeter size and begin secreting angiogenic factors such as VEGF (Folkman, Angiogenesis: An organizing principle for drug discovery. Nature Rev Drug Discovery. 6 (4): 273-286 (2007)). VEGF induced over-expression of TF in tumor-associated VECs is common in tumors. Hence, TF on the tumor-associated VECs should serve as the universal target. Since fVIIa is an endogenous ligand for TF, the conjugate is able to seek out microscopic metastases before they can be visualized by any conventional methods, delivering a drug such as EF24 to those cells and subsequently kill them. Drug resistance is a major problem in cancer treatment. Emergence of resistance is due in part to genetic instability, heterogeneity, and high mutation rate of tumor cells. In contrast, endothelial cells are genetically stable, homogeneous, and rarely mutative. Therefore, anti-angiogenic therapy using the conjugate directed against the tumor-associated TF-expressing endothelial cells should induce little or no drug resistance.

Upregulation of TF expression has been observed in many cancers, including bladder, breast, colorectal, gastric, hepatocellular, non-small cell lung, ovary, pancreatic, and prostate cancers, as well as glioma, and melanoma. It is worth noting that TF expression is not observed either in normal tissue or in tissue adjacent to the malignant tumor. The increased TF expression in a tumor may act as an angiogenic switch to enhance tumor angiogenesis and to convert the tumor to more malignant phenotypes. Concomitant expression of TF and VEGF has been demonstrated in a variety of tumors, including adenocarcinoma of the lung, breast, colorectal, hepatocellular carcinomas, and glioma. These observations imply the involvement of TF in tumor angiogenesis in both experimental tumors and human cancers. Higher levels of TF expression in tumors has been shown to correlate well with higher degree of tumor angiogenesis as indicated by an increase in VEGF and micro-vessel density (MVD), while the latter has been considered as an indicator for aggressive tumor progression, metastasis and poor prognosis. These revealing correlations have been demonstrated in gliomas, colorectal, hepatocellular, non-small cell lung, pancreatic and prostate cancers. Increased levels of VEGF appear to concurrently induce aberrant TF expression on VECs of the tumor vasculature. Within this setting, TF is a prime target for an anti-cancer drug delivery system that simultaneously attacks both tumor angiogenesis and the tumors. The results below demonstrate that the EF24-FFRck-fVIIa conjugate accomplishes both actions. The conjugate inhibits angiogenesis in both the rabbit cornea model and the Matrigel model in female athymic nude mice, and it reduces tumor size of human breast cancer xenografts in female athymic nude mice. The conjugate is cytotoxic only to TF-expressing cells in a dose-dependent manner, whereas EF24-FFRck (unconjugated to fVIIa) exhibited no such effect. The TF-dependent cytotoxicity of the EF24-FFRck-fVIIa conjugate is further demonstrated by the fact that the conjugate causes very little cell death to non-TF-expressing cells. These include normal breast cells and melanocytes, as well as normal HUVECs that do not express TF. The result strongly suggests that EF24-conjugated fVIIa binds to TF on the cell surface and the resulting complex is subsequently endocytosed into the target cells. Although it has not been shown directly for the EF24-FFRck-fVIIa conjugate, the data described below coupled with previous demonstrations of fVIIa/TF endocytosis by other investigators demonstrate compatibility with the hypothesis that the conjugate binds to TF on the cell surface and is subsequently internalized. Once inside the cells, it is likely that the complex is enzymatically cleaved to free EF24, resulting in its cytotoxic action

The physiological functions to be modulated include, but are not limited to, proliferation of the cells, for example reducing proliferation of the cells. Exemplary target include vascular endothelial cells, vascular smooth muscle cells, tumor cells, monocytes, and macrophages. In one embodiment, the target cell is a vascular endothelial cell. In yet another embodiment, the target cell is a vascular smooth muscle cell. The vascular endothelial cell can be selected from the group consisting of an isolated vascular endothelial cell, a capillary endothelial cell, a venal endothelial cell, an arterial endothelial cell and a neovascular endothelial cell of a tumor. In still another embodiment the target cell is a cultured cell.

The conjugates can also be administered to modulate a pathological condition in an animal or human by administering to an animal or human subject having a pathological condition an effective dose of a composition containing a cytotoxic compound-protein conjugate, thereby reducing the proliferation of a target cell capable of expressing surface-bound marker, and thereby modulating the pathological condition of the patient subject. Exemplary pathological conditions include cancer, hypercoagulapathy, restenosis, diabetic retinopathy, rheumatoid arthritis and inflammation associated with skin disorders. Exemplary cancers include leukemia, breast cancer, lung cancer, liver cancer, melanoma and prostrate cancer.

For example, the compositions may be antiangiogenic, wherein reducing proliferation of a target cell reduces angiogenesis and, in another embodiment, reducing angiogenesis causes a reduction in a tumor.

The regimen for any patient to be treated with a pharmaceutical composition mentioned herein should be determined by those skilled in the art. The daily dose to be administered in therapy can be determined by a physician and will depend on the particular compound employed, on the route of administration and on the weight and the condition of the patient. The efficacy of the curcuminoids suitable for use in the present invention as cytotoxic agents effective against cancer cells is described in WO 01/40188 incorporated herein in its entirety.

The amount of active ingredient to be administered will generally be that amount of the curcuminoid derivatives thereof which produces a therapeutic effect. Generally, out of one hundred percent, this amount will range from about 0.1 percent to about 99.5 percent of active ingredient, preferably from about 5 percent to about 70 percent, most preferably from about 10 percent to about 30 percent.

A. Cancers

The cytotoxic effects of the curcumin-FFRck-fVIIa constructs were tested on human prostate cancer cells (Example 8), breast cancer and melanoma cells (Example 9), umbilical cord vascular endothelial cells (HUVECs) (Example 11) and murine vascular endothelial cells immortalized by transfection of SV40 large T antigen (MS-1 Cells). MS-1 cells are regarded as benign because the cells, when in nude mice, remain as small tumors a few millimeters in diameter during the entire life span of the mice, and do not metastasize. Normal HUVEC cells induced to express high-levels of tissue factor by exposure to phorbol ester are susceptible to the cytotoxic effect of the EF24-FFR-ck-fVIIa conjugate, as shown in Example 11, below.

B. Skin Disorders

For the treatment of skin disorders, the angiogenesis inhibitors of the present invention are preferably administered systemically. For treatment of certain disorders, however, the curcuminoid-tetherlinker-fV-IIa may be applied topically in diseases or pathologic conditions of the skin, or locally in other tissues, to treat cancer, pre-malignant conditions and other diseases and conditions in which angiogenesis occurs.

The administration of these agents topically or locally may also used to prevent initiation or progression of such diseases and conditions. For example, a curcuminoid formulation may be administered topically or by instillation into a bladder if a biopsy indicated a pre-cancerous condition or into the cervix if a Pap smear was abnormal or suspicious.

The angiogenesis inhibiting formulation is administered as required to alleviate the symptoms of the disorder. Assays can be performed to determine an effective amount of the agent, either in vitro or in vivo. Representative assays are described in the examples provided below. Other methods are known to those skilled in the art, and can be used to determine an effective dose of these and other agents for the treatment and prevention of diseases or other disorders as described herein.

C. Restenosis

Recent advances in the treatment of coronary vascular disease include the use of mechanical interventions to either remove or displace offending plaque material in order to re-establish adequate blood flow through the coronary arteries. Despite the use of multiple forms of mechanical interventions, including balloon angioplasty, reduction atherectomy, placement of vascular stents, laser therapy, or rotoblator, the effectiveness of these techniques remains limited by an approximately 40% restenosis rate within 6 months after treatment.

Restenosis is thought to result from a complex interaction of biological processes including platelet deposition and thrombus formation, release of chemotactic and mitogenic factors, and the migration and proliferation of vascular smooth muscle cells into the intima of the dilated arterial segment. The inhibition of platelet accumulation at sites of mechanical injury can limit the rate of restenosis in human subjects. Inhibition of platelet accumulation at the site of mechanical injury in human coronary arteries is beneficial for the ultimate healing response that occurs. While platelet accumulation occurs at sites of acute vascular injuries, the generation of thrombin at these sites may be responsible for the activation of the platelets and their subsequent accumulation.

The modified fVIIa is able to bind to cell-surface tissue factor but has no enzymatic activity. It will, however, act as a competitive antagonist for wild-type fVIIa, thereby inhibiting the subsequent steps in the extrinsic pathway of coagulation leading to the generation of thrombin.

Modified fVIIa molecules that maintain tissue factor binding, inhibit platelet accumulation at the site of vascular injury by blocking the production of thrombin and the subsequent deposition of fibrin.

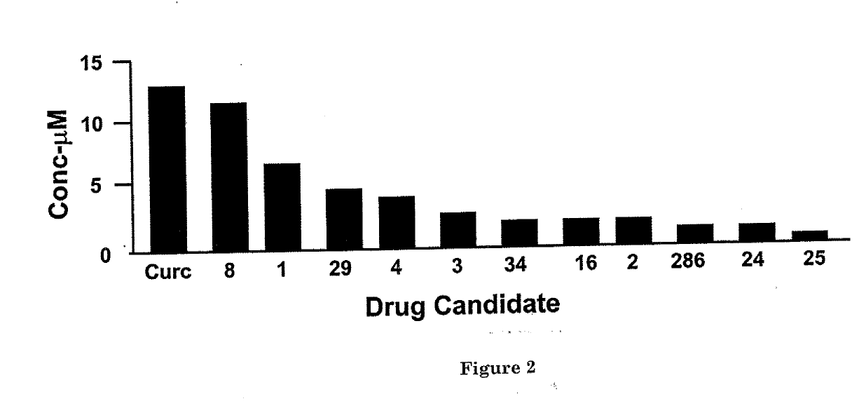

The curcuminoid-linker-fVIIa conjugates block thrombin generation and limit platelet deposition at sites of acute vascular injury, and therefore are useful for inhibiting vascular restenosis. The compositions may further inhibit restenosis by internalization by proliferating endothelial or smooth muscle cells, thereby delivering curcuminoids such as, but not limited to, EF24, to the cytoplasm of a target cell. The curcuminoids may then directly kill the target cell, as shown in FIG. 2 wherein various candidate curcuminoids including EF24 were added to endothelial cells immortalized with the Ras gene, thereby reducing or eliminating restenosis.

Thus, the compositions and methods have a wide variety of uses. For example, they are useful in preventing or inhibiting restenosis following intervention, typically mechanical intervention, to either remove or displace offending plaque material in the treatment of coronary or peripheral vascular disease, such as in conjunction with and/or following balloon angioplasty, reductive atherectomy, placement of vascular stents, laser therapy, rotoblation, and the like. The compounds will typically be administered within about 24 hours prior to performing the intervention, and for as much as 7 days or more thereafter. Administration can be by a variety of routes as further described herein. The preferred route will be direct delivery to a blood vessel, possibly close to the site of restenosis or tissue damage for rapid and specific delivery to tissue factor-bearing cells. The compounds can also be administered systemically or locally for the placement of vascular grafts (e.g., by coating synthetic or modified natural arterial vascular grafts), at sites of anastomosis, surgical endarterectomy (typically carotid artery endarterectomy), bypass grafts, and the like. The modified fVIIa also finds use in inhibiting intimal hyperplasia, accelerated atherosclerosis and veno-occlusive disease associated with organ transplantation, e.g., following bone marrow transplantation.

The curcuminoid-linker-fVIIa conjugates are particularly useful in the treatment of intimal hyperplasia or restenosis due to acute vascular injury. Acute vascular injuries are those which occur rapidly (i.e. over days to months), in contrast to chronic vascular injuries (e.g. atherosclerosis) which develop over a lifetime. Acute vascular injuries often result from surgical procedures such as vascular reconstruction, wherein the techniques of angioplasty, endarterectomy, atherectomy, vascular graft emplacement or the like are employed. Hyperplasia may also occur as a delayed response in response to, e.g., graft emplacement or organ transplantation. Since conjugated fVIIa is more selective than heparin, generally binding only tissue factor which has been exposed at sites of injury, and because modified fVII does not destroy other coagulation proteins, it will be more effective and less likely to cause bleeding complications than heparin when used prophylactically for the prevention of deep vein thrombosis. The dose of modified fVII for prevention of deep vein thrombosis is in the range of about 50 μg to 500 mg/day, more typically 1 mg to 200 μg/day, and more preferably 10 to about 175 μg/day for a 70 kg patient, and administration begin at least about 6 hours prior to surgery and continue at least until the patient becomes ambulatory. The dose of the curcuminoid-fVIIa conjugates of the present invention in the treatment for restenosis will vary with each patient but will generally be in the range of those suggested above.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims. The present invention is further illustrated by the following examples, which are provided by way of illustration and should not be construed as limiting.

### EXAMPLES

### Example 1

### Purification of Factor VII (fVIIa)

Human purified factor VIIa suitable for use in the present invention is preferably made by DNA recombinant technology, e.g. as described by Hagen et al., Proc. Natl. Acad. Sci. USA 83: 2412-2416, (1986) or as described in European Patent No. 0 200 421. Factor VIIa produced by recombinant technology may be authentic factor VIIa or a more or less modified factor VIIa provided that such factor VIIa has substantially the same biological activity for blood coagulation as authentic factor VIIa. Such modified factor VIIa may be produced by modifying the nucleic acid sequence encoding factor VII either by altering the amino acid codons or by removal of some of the amino acid codons in the nucleic acid encoding the natural fVIIa by known means, e.g. by site-specific mutagenesis.

Factor VII may also be produced by the methods described by Broze & Majerus, J. Biol. Chem. 255 (4): 1242-1247, (1980) and Hedner & Kisiel, J. Clin. Invest. 71: 1836-1841, (1983). These methods yield factor VII without detectable amounts of other blood coagulation factors. An even further purified factor VII preparation may be obtained by including an additional gel filtration as the final purification step. Factor VII is then converted into activated fVIIa by known means, e.g. by several different plasma proteins, such as factor XIIa, IXa or Xa. Alternatively, as described by Bjoern et al. (Research Disclosure, 269 September 1986, pp 564-565), factor VII may be activated by passing it through an ion-exchange chromatography column, such as Mono QR™ (Pharmacia Fine Chemicals) or similar column.

### Example 2

### Synthesis of Curcumin Analogs and Coupling of EF24 and FFRck Using a Succinate Tether

Descriptions and synthetic preparations of a series of monocarbonyl curcumin analogs useful in the present invention has been described in PCT Application Serial No. 01/40188 incorporated herein by reference in its entirety.

Synthesis of the conjugate of fVIIa protein and the drug molecule, EF24-FFRck-fVIIa proceeded in three steps. First, an appropriate derivative of EF24 was developed that permitted attachment to the FFR tripeptide. To synthesize EF24, piperidone.hydrate.HCl (2.2 g, 14 mmol) was suspended in glacial CH₃COOH (60 ml). The suspension was saturated with HCl gas until the solution became clear, and then treated with solid 2-fluorobenzaldehyde (5.0 g, 40 mmol). The reaction mixture was stirred at ambient temperature for 48 hrs. The precipitated solid was collected by filtration, washed with cold absolute ethanol, and dried in vacuo to give a bright-yellow crystalline solid (EF24, 4.27 g, 80% yield).

Of several compounds examined, the succinic acid derivative aa (86% yield) was suitable since it retained 50% of the activity of EF24 in cell cytotoxicity assays. To synthesize the succinyl derivative aa of EF24, to a solution of EF24 (0.16 g, 0.5 mmol) in anhydrous CH₂Cl₂ (6 ml) was added succinic anhydride (0.057 g, 0.5 mmol) and Et₃N (101 mg, 1 mmol). The mixture was stirred at room temperature for 3 hrs, diluted with CH₂Cl₂, washed twice with saturated NaHCO₃ (2×10 ml) and brine, dried over anhydrous Na₂SO₄, and relieved of solvent by evaporation. The resulting solid was purified by flash chromatography using benzene/acetone/acetic acid (27:10:0.5) as the eluant to obtain the yellow solid aa (852 mg, mp 145° C., 86% yield). The reaction scheme is shown below.

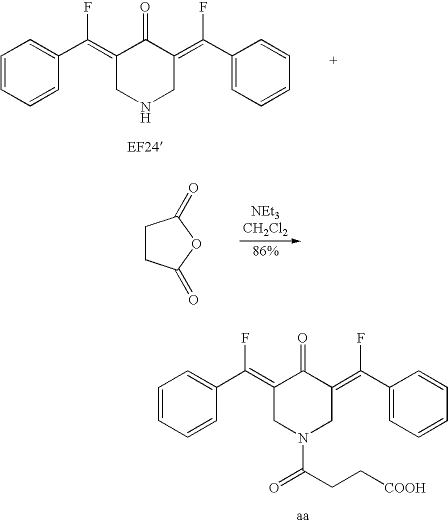

Second, the FFR-ck peptide linker was assembled as shown below. For this step, commercially available Boc-Arg(Mtr)-OH (ab 122 mg, 0.25 mmol) was dissolved in THF (2 ml) and allowed to react with isopropyl chloroformate (1.0 M in toluene, 0.25 ml, 0.25 mmol) in the presence of N-methylmorpholine (25 mg, 0.25 mmol) for 4 hrs at −20° C. The mixture was filtered, and the filtrate was added to 4 ml of ethereal diazomethane. After stirring the reaction solution for 1 hr at 0° C., the solvent was evaporated to obtain the crude product as white needles. These were purified by chromatography using ethyl acetate as the eluant to obtain a white solid, ac (75 mg, 59% yield). The reaction scheme is shown below:

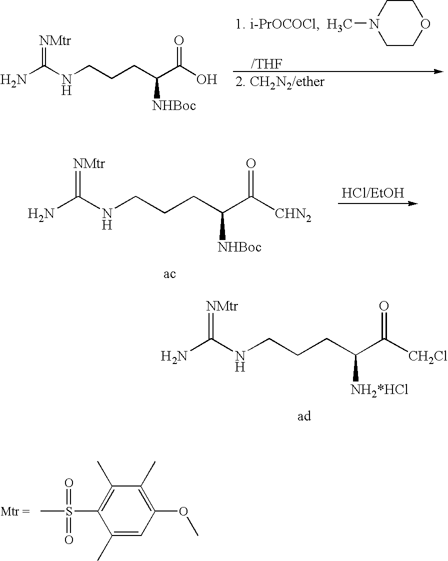

N-Boc-Phe-Phe-OH af (197 mg, 0.4 mmol) was allowed to react with N-methylmorpholine (40 mg, 0.4 mmol) and isopropyl chloroformate (1.0 M in toluene, 0.4 ml, 0.4 mmol) for 10 mins at −20° C. Cold THF (5.72 ml) containing N-methylmorpholine (40 mg, 0.4 mmol) was added to the mixture which was immediately added to Arg(Mtr)CH₂Cl*HCl (ad, 200 mg, 0.4 mmol) dissolved in DMF (0.92 ml). After stirring for 1 hr at −20° C. and 2 hrs at room temperature, THF (5.6 ml) was added and the mixture was filtered. The filtrate was evaporated and the solid residue purified by column using EtOAc/hexanes (4:1) as the eluant. A white solid was obtained, ag (245 mg, 75% yield). The reaction scheme is shown below.

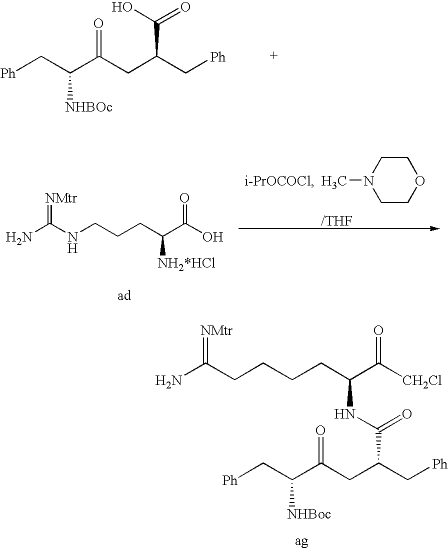

Compound ag (0.05 mmol, 42.5 mg) was dissolved in EtOAc (0.16 ml) and allowed to react with methanolic HCl (0.85 mmol) at room temperature for 3.5 hrs, washed with NaHCO₃ (aq), extracted with CH₂Cl₂ (2×10 ml) and dried over MgSO₄ and filtered. Evaporation of the solvent furnished a white solid, FFR-ck (ah, 40 mg).

To a mixture of ah (24 mg, 0.032 mmol) and aa (12 mg, 0.03 mmol) in CH.sub.2Cl.sub.2 (0.6 ml) was added DCC (6.18 mg, 0.03 mmol). After stirring overnight at room temperature, filtration and evaporation of the solvent, and purification by flash chromatography using ethyl acetate as the eluant ai (17 mg, 49% yield) was obtained. Compound ai (34 mg, 0.03 mmol) was dissolved in 95% aqueous TFA (0.95 ml) with thioanisole (0.05 ml). The resulting dark solution was stirred for 48 hrs at room temperature and then concentrated under vacuum. The resulting solid was triturated with ether, recrystallized and dried under a vacuum to supply compound aj (EF24-FFR-ck) (12 mg, 45% yield).

The reaction scheme is shown below.

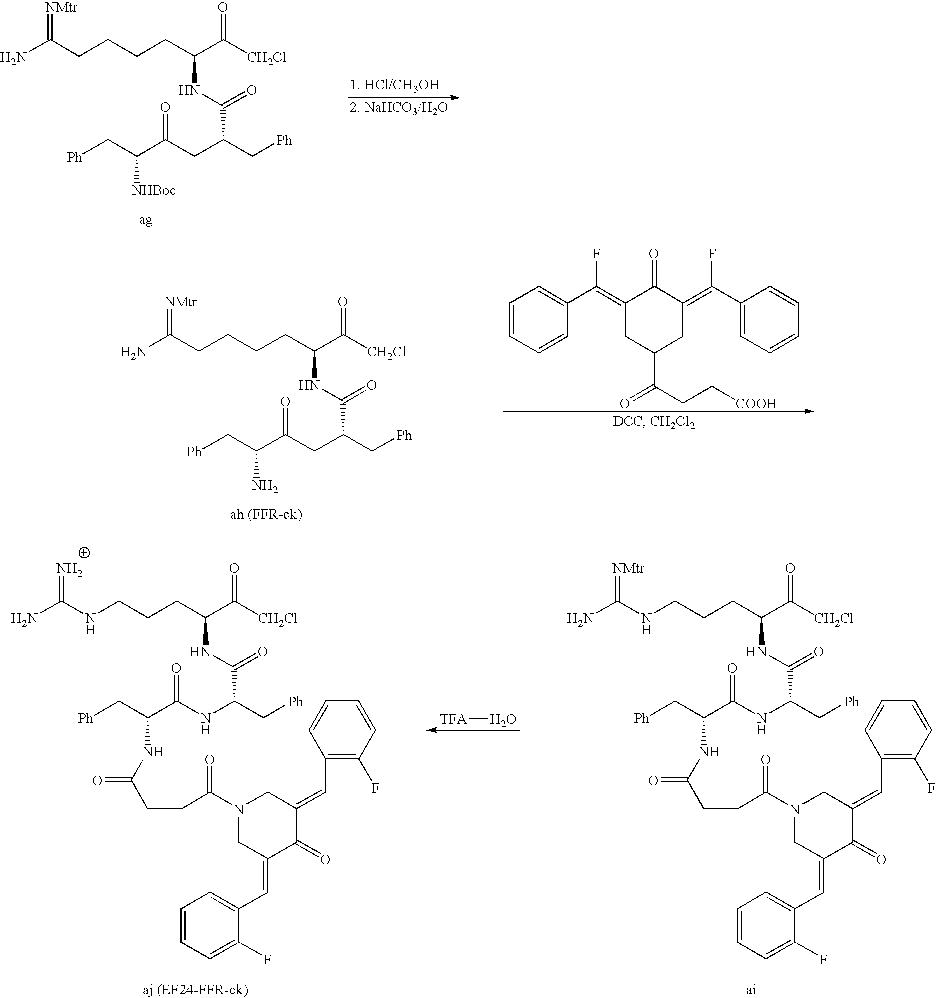

### Example 3

### Coupling of EF24-FFRck (aj) and fVIIa

Method 1: Recombinant fVIIa (250 μg) was resuspended in 0.5 ml of distilled water and dialyzed in 1 liter of 1 mM Tris HCl, pH 8.0 at 4° C. overnight. Forty-fold molar excess of EF24-FFRck (aj) synthesized as described in Example 2 above, in 0.25 ml of DMSO was added to a final concentration of 400 μM. The mixture was covered with aluminum foil (EF24 is photosensitive) and incubated at room temperature overnight in darkness. The reaction mixture was centrifuged at 16,000 rpm at 4° C. for 20 minutes in a Sorvall centrifuge to precipitate unbound EF24-FFRck and separate it from EF24-FFRck-fVIIa. The supernatant was further dialyzed in 100 ml of sterile cell culture medium containing 10% fetal bovine serum, penicillin (100 units/ml) and streptomycin 100 μg/ml) at 4° C. overnight. EF24-FFRck-fVIIa equilibrated with the culture medium was added to cells in wells of a 96-well plate.

Method 2: (1). Factor VIIa (fVIIa) will be dialyzed against 1.0 mM Tris HCl, pH 7.4 at 4° C. overnight. (2) EF24-FFRck will be dissolved in 100% DMSO. (3) fVIIa per ml and EF24-FFRck per 0.25 ml will be mixed at a molar ratio 1:13.2 and gently stirred for 2 hrs at room temperature. (4) an additional EF24-FFRck per 0.25 ml (at a molar ratio of 1:13.2) will be added to the reaction mixture to make the final molar ratio 1:40 and continue the coupling reaction overnight at room temperature. (5) the coupled product EF24-FER-ck-fVIIa will be separated from uncoupled EF24-FFRck by column chromatography and 0.5 ml fractions collected. (6) a protein peak (fVIIa) will be determined by reading fractions at OD₂₈₀ and the Bradford protein determination (Bio-Rad) (7) active fractions will be pooled.

Method 3: Recombinant human fVIIa was first dialyzed in a buffer containing 1 mM Tris-HCl (pH 7.5) and 0.1% Tween-80 to prevent EF24 precipitation at high buffer concentrations and in the presence of salt. EF24-FFRck was solubilized in 100% DMSO and added slowly to an equal volume of the above dialyzed fVIIa solution. The conjugation was continued at 4° C. for 64 h in total darkness to prevent photo-inactivation of EF24. The resulting mixture was then dialyzed at 4° C. against the same buffer to remove DMSO and free EF24-FFRck. The dialysis membranes used (Spectra/Pro) have a cut-off pore size of 13 kDa. Since unbound EF24-FFRck has a molecular weight of 894 Da, most of the unbound EF24-FFRck should be removed after extensive dialysis. Subsequently, the conjugate was concentrated using a Centricon-30 which excludes molecules with MW equal or less than 30,000 Daltons. This concentration step further filters out residual free EF24-FFRck. The resulting conjugate was then assayed for protein content using a Bio-Rad protein assay.

The concentration of EF24-FFRck-bound fVIIa was estimated based on the protein concentration since 1 molecule of EF24-FFRck binds 1 molecule of fvlIa. Fluorescein-FPRck (0.1 mg/20 μl in DMSO) was purchased from Innovative Research, Inc. The compound was dissolved in 1 ml of DMSO and conjugated to fVIIa at a molar ratio of fluorescein-FPRck to fVIIa of 40:1 as described. The conjugate was concentrated and the tube was wrapped in aluminum foil and stored at 4° C.

### Example 4

### Mass Spectroscopic Examination of the Coupled EF24-FFRck (al) to fVIIa

The native fVIIa and EF24-FFRck-fVIIa conjugate were dialyzed against 10 mM Tris-HCl, pH 7.5 and purified by microbore reversed-phase HPLC (model 140A/785A, Applied Biosystems) using an mRP-Hi-Recovery Protein Column (2.1×75 mm, Agilent) equilibrated in 0.1% aqueous trifluoroacetic acid (TFA) containing 20% (v/v) acetonitrile and eluted at 25° C. using a linear gradient (1% per min) of acetonitrile in 0.08% TFA. The column effluent was monitored at 214 nm, and the fractions were manually collected. The fractions were evaporated in a Speed-Vac concentrator, reconstituted in 5 μl of water/methanol/acetic acid (25:25:1, v/v) and analyzed by nano-ESI-MS using model QSTAR-XL quadrupole-TOF mass spectrometer (Applied Biosystems).

The masses obtained from nano-ESI-MS analysis for fVIIa and the EF24-FFRck-fVIIa conjugate were 45,130 Da and 45,984 Da, respectively, with a difference of 854 Da. The expected mass difference is 857 Da, indicating an attachment of one molecule of EF24-FFR to one molecule of fVIIa. It was demonstrated that His-193 within the catalytic triad of the fVIIa serine protease active site is the amino acid residue to which FFR-ck covalently binds (Williams et al. 1989). Since only one molecule of EF24-FFRck is bound to fVIIa, it is most likely that EF24-FFRck is also bound to His-193 on fVIIa in a manner similar to FFRck.

### Example 5

### Binding Activity of Molecule-FFRck-fVIIa Conjugates to Soluble TF

A 96-well ELISA plate was coated with 100 μl of soluble TF in phosphate buffered saline (PBS) at 5 pmoles per well. The plate was kept at 4° C. overnight. The wells were then treated with blocking buffer (Protein-free T-20 Blocking buffer, product #37573, Pierce Chemical Company, Rockford, Ill.) for 2 hours. The EF24-FFRck-fVIIa conjugate or the fluorescein-FPRck-fVIIa conjugate with 2-fold serial dilutions in blocking buffer was applied to the wells. The highest amount of fVIIa or the fVIIa conjugates was 50 pmoles per well. The plate was kept at 37° C. for 2 hours, and then washed 5 times with wash buffer (PBS containing 0.05% Tween-20). Anti-fVIIa mAb (AA-3) was conjugated with biotin using a biotinylation kit (EZ-link NHS-biotin reagents, product #21336, Pierce). Biotinylated anti-fVIIa mAb (AA-3) diluted with blocking buffer was applied to the wells and allowed to stand at room temperature for 1 hour. After washing, horse radish peroxidase (HRP)-conjugated streptavidin (product #N100, Pierce) diluted in blocking buffer was then added to the wells and allowed to react for 1 hour. After washing, 100 μl of HRP substrate (1-Step Turbo TMB, product #34022, lot #HH10555-1A, Piece) was added to each well. After 15 minutes, 100 μl of stop buffer was added to each well. Absorbance was measured at 450 nm, Binding activities were expressed as % of binding by the highest amount of fVIIa or conjugates.

Binding of Conjugates to TF In Vitro

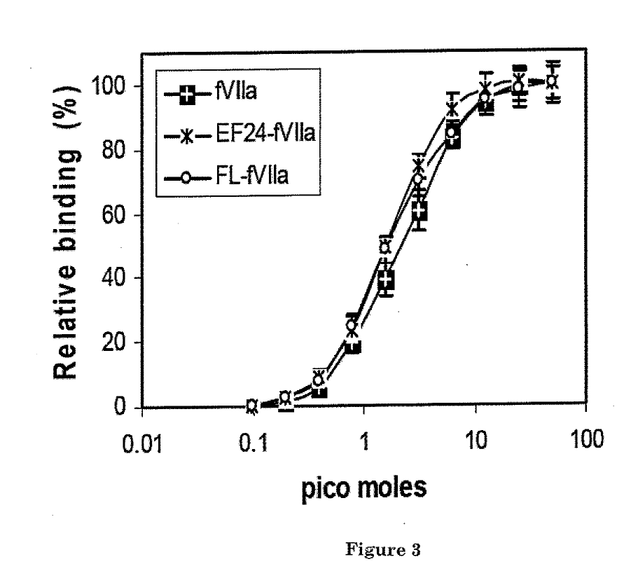

To further evaluate the conjugates, we compared the binding property of the conjugates with the native fVIIa to TF. The results show that 50% binding of fVIIa, the EF24-FFRck-fVIIa conjugate and the fluorescein-FPRck-fVIIa conjugate to TF corresponds to 2.3, 1.6, and 1.6 pmoles, respectively (FIG. 3). The native fVIIa and the two conjugates bind to TF similarly. This result is consistent with other published data which show that binding affinity of FFRck-fVIIa to TF is higher than the native fVIIa.

TF-Dependent Endocytosis of the Fluorescein-FPRck-fVIIa Conjugate

To test whether fVIIa can specifically deliver the conjugated drug into target cells, we used the fluorescein-FPRck-fVIIa conjugate since the binding affinities of EF24-FFRck-fVIIa and fluorescein-FPRck-fVIIa are essentially the same. Thus, we incubated MDA-MB-231 cells expressing high TF (Shoji et al. 1998, Zhou et al. 1998), normal HUVECs expressing no TF and TPA-treated HUVECs expressing moderate TF with 10 nM of the fluorescein-FPRck-fVIIa. Endocytosis of the conjugate was detected by fluorescent imaging. The result showed that the fluorescein-FPRck-fVIIa conjugate was endocytosed by breast cancer cells and TPA-treated HUVECs, but very weak fluorescence was detected with the non-TPA-treated (normal) HUVECs. The difference in fluorescence intensity observed in breast cancer cells and TPA-treated HUVECs reflects the level of TF expression in those cells.

TF-Dependent Cytotoxicity of the EF24-FFRck-fVIIa Conjugate In Vitro

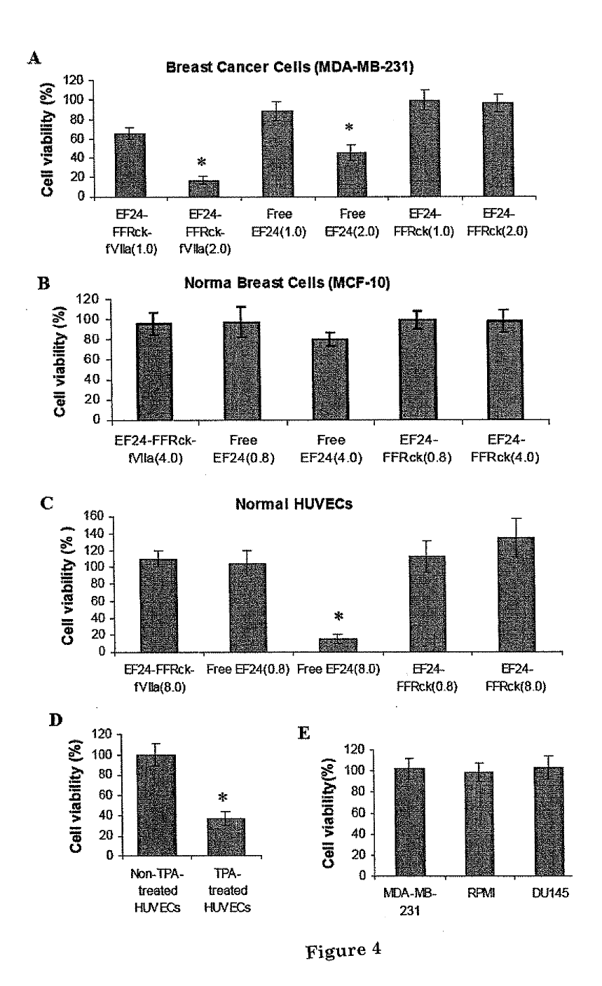

To demonstrate that the cytotoxicity of the EF24-FFRck-fVIIa conjugate is TF-dependent, we tested the conjugate on TF-expressing and non-TF-expressing cells (FIGS. 4A-E). The TF-expressing cells included MDA-MB-231, RPMI-7951, DU-145 producing high levels of TF and VEGF (TF, 10,690+650 pg/106 cells; VEGF, 30,511+5,748 pg/106 cells) and HUVECs pre-treated with or without phorbol ester (TPA) for TF induction. The non-TF-expressing cells included the normal human breast cell line, MCF10 normal melanocytes (data not shown), and normal HUVECs. The results show that the EF24-FFRck-fVIIa conjugate significantly decreases the viability of the TF-expressing cells [MDA-MB-231 (FIG. 4A), RPMI-7951 and DU-145 (data not shown)] in a dose-dependent manner, while the conjugate had very little effect on non-TF-expressing cells, including normal breast cells (FIGS. 4B and 4C) and normal melanocytes (data not shown). The conjugate was also significantly more cytotoxic to the TF-expressing VECs than the non-TPA-treated HUVECs (FIG. 4D). Unconjugated EF24 exhibits non-specific cytotoxicity to all cells regardless of the extent of TF expression (FIGS. 4A-4C). EF24-FFRck, unconjugated to fVIIa, has no effect on cell viability for the cell lines tested due to its lack of binding to cell-surface TF. That is, it cannot be endocytosed into the cells like the conjugate (FIGS. 4A-C). The drug carrier FFRck-fVIIa is not cytotoxic to any of the TF-expressing cancer cells including MDA-MB-231, RPMI-7951, and DU-145 (FIG. 4E).

### Example 6

### Inhibition of VEGF-A Induced Angiogenesis in Matrigel Plugs in Mice by the EF24-FFRck-fVIIa Conjugate

To assess the inhibition of VEGF-A induced angiogenesis by the EF24-FFRck-fVIIa conjugate in a Matrigel model in mice, 0.3 ml of Matrigel (Cat. 356231, BD Biosciences, Mass.) containing 600 ng of VEGF-A (VEGF-165, Cat. 293-VE-010, R & D, Minneapolis, Minn.) was implanted subcutaneously in the flanks of female athymic nude mice. On days 12 and 14 after the Matrigel implantation, 0.1 ml of the EF24-FFRck-fVIIa conjugate (containing 50 μM EF24) or vehicle was administered intravenously. On day 18, Matrigel plugs were collected. Hemoglobin content was determined in a pool of 5 randomly selected Matrigels from the treated and the control mice by the Drabkin method (Cat. D59410, Sigma Chemical, St. Louis, Mo.) according to the manufacturer's instructions.

We used two systems to test the hypothesis that the EF24 conjugate should bind TF and kill the TF-expressing VECs when VEGF induces TF expression on VECs in vivo. The first system is a Matrigel plug assay in mice. The Matrigel plugs from the EF24 conjugate-treated mice appear less bloody than those from the untreated mice. To quantify the level of inhibition of angiogenesis by the conjugate, the Drabkin assay was performed to compare the levels of hemoglobin in Matrigel plugs from the conjugate-treated and vehicle-treated mice. The result shows a 60% reduction in hemoglobin content in Matrigel plugs from the conjugate-treated mice as compared to those from the untreated animals.

### Example 7

### Inhibition of VEGF-A Induced Rabbit Cornea Angiogenesis by the EF24-FFRck-fVIIa Conjugate

Assay for rabbit cornea angiogenesis was carried out as described by Shan et al). VEGF-A pellets were prepared as previously described. Hydron polymer (IFN Sciences, New Brunswick, N.J.) was dissolved in absolute ethanol at 12% (w/v). VEGF-A solution was prepared at 200 ng/μl with sterile PBS containing 0.1% BSA. Sucralfate (sucrose octasulfate aluminum complex; Sigma) solution was prepared with sterile PBS at 100 μg/μl and stored at 4° C. Hydron pellets were prepared by mixing equal volumes of Hydron gel and Sucralfate solution containing VEGF-A. Each pellet for the corneal pocket assay contained 200 ng of VEGF-A and 20 μg of Sucralfate in 3 μl of casting gel. The pellets were prepared the day before corneal surgery in a laminar flow hood under sterile conditions. Dried discs of uniformed size (2 mm in diameter) were chosen for experiment.

To implant VEGF pellets in rabbit cornea, 10 rabbits were anesthetized by intramuscular (i.m.) injection of a mixture of 0.5 ml of ketamine (500 mg/5 ml/vial, Abbott Laboratories, Abbott Park, Ill.) and 0.5 ml of xylazine (20 mg/ml/vial, Vetus, Bachem Bioscience, King of Prussia, Pa.). A small incision of 1-2 mm was made on the cornea near the limbus of each eye using a diamond knife to create a pocket. A pellet containing VEGF-A was inserted into the pockets bilaterally. After the procedure, eye drops (containing neomycin, polymyxin B and garamycin) were given three times per day for 10 days. Finally, two rabbits that each exhibited good neovascularization were used for three treatment regimens, the EF24-FFRck-fVIIa conjugate, free EF24, and vehicle.

To apply treatments through the common carotid artery, Rabbits were anesthetized with an intramuscular injection of 2-3 ml of the mixture of ketamine/xylazine per animal and then a midline incision, approximately 6-8 cm, was made allowing the skin to be reflected laterally to expose the common carotid artery. One ml each of the EF24-FFRck-fVIIa conjugate solution (containing 50 μM EF24), free EF24 (50 μM) or vehicle (1 mM Tris-HCl, pH 7.5, containing 0.1% Tween-80) was injected into the common carotid artery, bilaterally. Afterwards, a pre-prepared surgicell-covered Q-tip was held down on the injection site for 3 min, with the surgicell remaining in place to maintain hemostatic pressure. The incision was closed with two layers of 2-0 vicryl (polysorb) and followed by closing the skin with a Michel clip. The area was cleaned with 70% alcohol, then the entire surgical site was dabbed with Betadine. After the procedure, Buprenex (0.3 mg/ml) (Reckitt Benckiser) was administered intramuscularly.

To quantify the extent of angiogenesis, blood vessels in rabbit corneas were traced onto transparent films directly from a computer screen of the rabbit eyes images. The number and length of blood vessels were quantified by using an NIH program Image-J. Each eye before treatment with the EF24-conjugate or vehicle served as a control (100%) 4 days after treatment.

One ml of the EF24-FFRck-fVIIa conjugate, free EF24 or vehicle was injected into both sides of the common carotid artery in order to deliver the drug conjugate directly to the corneal blood vessels before passing through the liver. The blood vessels on the rabbit cornea were monitored and photographed daily using a digital camera. With the treatment of the EF24-FFRck-fVIIa conjugate, the average number of the corneal blood vessels is significantly reduced to 39% of the pre-treatment level (average 39.0±10.3, p<0.01, n=4), whereas in the control group, the reduction is not significant (average 81.8±35.4, p: NS, n=4). Free EF24 of the same concentration as the conjugate did not show obvious inhibition of angiogenesis (data not shown).

### Example 8

### EF24-FFRck-fVIIa Binds Only TF Via fVIIa and Kills Human Prostate Cancer Cell Lines

Tissue factor (TF) and vascular endothelial growth factor (VEGF) levels expressed by DU 145 and PC3 prostate cancer cell lines were measured by ELISA, as shown in Table 1 below. High TF and VEGF levels were found in DU145 cells.

TABLE 1
Tissue factor (TF) and vascular endothelial growth factor (VEGF)
ELISA in DU145 and PC3 prostate cancer cell lines. High TF and
VEGF levels in DU145 cells. Values indicate Mean ± S.D.

| | |
|---|---|
| TF (pg/ml) | VEGF (pg/ml) |

| DU-145 | PC-3 | DU-145 | PC-3 |
|---|---|---|---|
| 10690 ± 650 | 230 ± 16 | 30511 ± 5748 | 2186 ± 307 |

DU145 cells were plated with 2.times.10⁴ cells/100 μl/well in a 96 well plate and cultured overnight. The cells were cultured for 48 hrs. Cultures were terminated by adding 40% TCA to a final concentration of 10%. Cells were fixed in TCA at 4° C. for 1 hr, washed with tap water 5 times and air dried. Sulforhodamine B (SRB) solution (100 μl) at 0.4% (w/v) in 1% acetic acid was added to each well, and the cells were incubated for 10 mins at room temperature. After staining, unbound dye was removed by washing five times with 1% acetic acid and air dried. Bound dye was subsequently solubilized with 200 μl of 10 mM Trizma base, and the absorbance was read on an automated plate reader at a wavelength of 490 nm. Assays were performed in triplicate or quadruplicate. An asterisk (*) indicates p<0.0001 by the Student t-test (two-tailed probability), The concentration of EF24-FFRck-fVIIa was estimated based on protein concentration.

EF24-FFRck alone does not kill any cells since it cannot bind the cell surface, as shown in Table 2.

TABLE 2
EF24-FFRck-fVIIa kills DU145, a Human Prostate Cancer Cell Line
which expresses Tissue Factor, SRB Viability Test. Values are Mean S.D.

| | O.D. 579 nm |
|---|---|
| Control (0.5% DMSO) | 0.370 ± 0.015 |
| EF24-FFRck-fVIIa, 0.8 pM | 0.333 ± 0.053 |
| EF24-FFRck-fVIIa, 8 pM | 0.111 ± 0.004* |
| EF24, 0.8 pM | 0.391 ± 0.041 |
| EF24, 8 pM | 0.053 ± 0.025* |
| EF24-FFRck, 0.8 pM | 0.389 ± 0.021 |
| EF24-FFRck, 8 pM | 0.383 ± 0.027 |

### Example 9

### EF24-FFRck-fVIIa Kills Human Breast Cancer and Melanoma Cells

TABLE 3
EF24-FFRck-fVIIa kills Human Breast Cancer (MDA-MB-231) and
Melanoma (RPMI-7951) NR Viability Test. Values indicate Mean ± S.E.

| | O.D. 570 nm | |
|---|---|---|
| | MDA231 | RPM17951 |
| Control (0.5% DMSO) | 0.193 ± 0.019 | 0.269 ± 0.019 |
| EF24-FFRck-fVIIa, 0.5 pm | 0.142 ± 0.010 | 0.292 ± 0.028 |
| EF24-FFRck-fVIIa, 2 pM | 0.041 ± 0.002* | 0.066 ± 0.002* |
| EF24, 1 pM | 0.172 ± 0.020 | 0.220 ± 0.023 |
| EF24, 2 pM | 0.109 ± 0.014* | 0.119 ± 0.018 |
| EF24-FFRck, 1 pM | 0.191 ± 0.013 | 0.253 ± 0.018 |
| EF24-FFRck, 2 pM | 0.171 ± 0.009 | 0.247 ± 0.020 |

Student t-test (two-tailed probability)(95% confident level)

The Neutral Red (NR) dye viability assay, instead of the Sulforhodamine B (SRB) assay, was used. In the NR viability assay, NR dye is taken up only by viable cells, while in the SRB viability assay, viable cells are fixed by trichloracetic acid (TCA) on the plate (thus, cells are killed), and the fixed cells are stained by SRB dye.

At the termination of culture, medium was removed and 200 μl of fresh, warm medium containing 50 μg of NR/ml was added to each well in a 96-well plate. Cells were incubated at 37° C. for 30 mins, followed by two washes with 200 μl of PBS. The NR taken up by cells was dissolved by adding 200 μl of 0.5N HCl containing 35% ethanol. The amount of the dye in each well was read at 570 nm by an ELISA plate reader.

### Example 10

### EF24-FFRck-fVIIa has No Effect on Normal Human Melanocytes and Normal Human Breast Luminal Ductal Cells

TABLE 4
EF24-FFRck-fVIIa has no effect on normal human melanocytes and
MCF10 (normal human breast luminal ductal cell line) which do
not express Tissue Factor: NR (neutral red dye) Viability Test.
Value are Mean ± S.D

| | O.D 570 nm | |
|---|---|---|
| | Melanocytes | Normal Breast Cells |
| Control (None) | 0.264 ± 0.023 | 0.106 ± 0.006 |
| DMSO (1%) | 0.261 ± 0.012 | 0.107 ± 0.012 |
| EF24-FFRck-fVIIa, 4 pM | 0.210 ± 0.005 | 0.096 ± 0.023 |
| EF24, 0.8 pM | 0.255 ± 0.009 | 0.104 ± 0.018 |
| EF24, 4 pM | 0.119 ± 0.009* | 0.091 ± 0.007** |
| EF24-FFRck, 0.8 pM | 0.252 ± 0.007 | 0.101 ± 0.013 |
| EF24-FFRck, 4 pM | 0.249 ± 0.015 | 0.113 ± 0.003 |

*p = 0.002,
**p = 0.031

Assays were performed essentially the same as for DU145 above.

### Example 11

### EF24-FFRck-fVIIa Does not Kill Normal HUVECs

TABLE 5
EF24-FFRck-fVIIa does not kill normal HUVECs that do not express
tissue factor: SRB Viability Test (NCI method). Mean ± S.D

| | O.D. 490 nm |
|---|---|
| Control (0.5% DMSO) | 0.119 ± 0.003 |
| EF24-FFRck-fVIIa, 0.8 pM | not done |
| EF24-FFRck-fVIIa, 8 pM | 0.370 ± 0.027^{a} |
| EF24, 0.8 pM | 0.136 ± 0.010 |
| EF24, 8 pM | 0.038 ± 0.010* |
| EF24-FFRck, 0.8 pM | 0.152 ± 0.026 |
| EF24-FFRck, 8 pM | 0.160 ± 0.038 |

*Student t-test (two-tailed probability)(95% confident level)
^{a}Cells were not washed before adding the SRB dye and therefore precipitated
EF24-FFRck-fVIIa adsorbed dye thereby giving a false elevated O.D. 490 nm value
EF24-FFRck-fVIIa does not kill normal HUVECs that do not express surface bound tissue factor.

### Example 12

### EF24-FFRck-fVIIa Kills HUVECs Induced to Express TF by 100 nM TPA

TABLE 6
EF24-FFRck-fVIIa kills HUVECs induced to express TF by 100 nM
TPA (phorbol ester) for 24 hrs prior to adding EF24-FFRck-fVIIa: SRB
Viability Test (NCI method). Mean ± S.D

| | TPA | O.D. 490 nm |
|---|---|---|
| EF24-FFRck-fVIIa, 0.6 pM | 0 | 0.170 ± 0.015 |
| EF24-FFRck-fVIIa, 0.6 pM | + | 0.059 ± 0.004* |

*Student t-test (two-tailed probability)(95% confident level)

### Example 13

### Novel Curcumin Analogs (A279L. A279U and EF-15) Are not Cytotoxic to Vascular Endothelial Cells

HUVECs, MS-1 cells and SVR cells were cultured to confluence and agents were incubated for 24 hrs. Cell viability was determined by Neutral Red assays. Among synthetic curcumin analogs, A279L, A279U and EF-15 were not cytotoxic at 20 μM. MS-1 cells were murine vascular endothelial cells which were immortalized by transfection of SV40 large T antigen but are non-maligant. However, when MS-1 cells were transfected with a ras mutant gene, cells were transformed to become malignant angiosarcoma cells, (SVR cells).

TABLE 7
Novel curcumin analogs (A279L. A279U and EF-15)
are not cytotoxic to vascular endothelial cells.

| | Neutral Red Viability Assay (% of Control) | | |
|---|---|---|---|
| | HUVECs | MS-1 Cells | SVR cells |
| DMSO | 100 | 100 | 100 |
| (0.1%) (control) | | | |
| Curcumin (1 μM) | 103 | 90 | 88 |
| (20 μM) | 21 | 3 | 4 |
| A279L (20 μM) | 97 | 90 | 90 |
| A279U (20 μM) | 92 | 96 | 92 |
| EF-15 (20 μM) | 100 | 95 | 100 |
| C.V.6 (1 μM) | 60 | 33 | 62 |
| (10 μM) | 26 | 10 | 3 |
| C.V.10 (1 μM) | 100 | 68 | 75 |
| (10 μM) | 17 | 7 | 5 |
| EF-2 (1 μM) | 24 | 8 | 20 |
| (10 μM) | 9 | 3 | 2 |
| EF-4 (1 μM) | 14 | 4 | 7 |
| (10 μM) | 15 | 4 | 6 |
| EF-17 (1 μM) | 93 | 87 | 75 |
| (10 μM) | 47 | 10 | 12 |
| EF-25 (1 μM) | 17 | 6 | 28 |
| (10 μM) | 17 | 6 | 4 |
| A283 (1 μM) | 38 | 21 | 37 |
| (10 μM) | 17 | 4 | 3 |
| A286 (1 μM) | 30 | 11 | 24 |
| (10 μM) | 17 | 8 | 4 |
| A287 (1 μM) | 80 | 43 | 67 |
| (10 μM) | 15 | 6 | 4 |

### Example 14

### Internalization of TF/FFR-ck-VIIa Complexes After Incubating Cells with Varying Concentrations of FFR-ck-fVIIa for 24 hrs

In three human cancer cell lines (high TF and VEGF producers), FFR-ck-VIIa alone caused internalization of TF into caveolac in the plasmalemma vesicles (Triton X-100 insoluble region of cell membrane) in a dose-dependent manner. FFR-ck-VIIa totally inhibited TF, which remained on the cell surface, to catalyze factor X to generate factor Xa. However, VEGF production and cell viability were not affected. In MDA-MB-231 cells, approximately 10 μM of FFR-ck-VIIa will be required to internalize 50% of TF-FFR-ck-VIIa complexes because MDA-MB-231 human breast cancer cells express greater level of TF than other cell lines.

TABLE 8
Effect of FFRck-fVIIa on cancer cells

| | | | |
|---|---|---|---|
| Tumor Cell Line | 0 | 100 | 1000 |

TF (nM) on the cell surface
FFR-ck-VIIa (nM)

| | | | |
|---|---|---|---|
| Hs294T | 6.0 ± 0.7 | 3.9 ± 0.6* | 2.5 ± 0.3* |
| RPMI7951 | 81.6 ± 4.5 | 38.7 ± 1.4* | 35.0 ± 6.2* |
| MDA-MB-231 | 624.8 ± 42.0 | 465.5 ± 17.7* | 488.9 ± 1.6* |

Percentage relative to 0 nM FFR-ck-VIIa control

| | | | |
|---|---|---|---|
| Hs294T | 100 | 65* | 42* |
| RPMI7951 | 100 | 76* | 48* |
| MDA-MB-231 | 100 | 91* | 80* |

Percentage internalized relative to 0 nM FFR-ck-VIIa control

| | | | |
|---|---|---|---|
| Hs294T | 0 | 35 | 58 |
| RPMI7951 | 0 | 24 | 52 |
| MDA-MB-231 | 0 | 9 | 20 |

Values of TF indicate mean ± S.D. of triplicate determinations.
*Statistically significantly different from control values (p < 0.05).

### Example 15

### Dissociation of Chemical Linkage Between Curcumin or Its Analogs and FFR-ck-VIIa (or YGR-ck-VIIa) Inside the Cells

1. Physical Analysis by HPLC Chromatography: Coupled compound such as EF24-FFR-ck-VIIa will be added to a confluent monolayer of cancer cells at an appropriate concentration and incubated for about 2-6 hours. Supernatants will be stored at −20° C. for VEGF ELISA assay. To dissociate surface-bound analog-FFR-ck-VIIa from TF, cells will be harvested with a rubber policeman and resuspended in 200 μl of ice-cold phosphate buffered saline (PBS)/HCl (pH 3.0) for 1 min at 0° C. The cells will be spun for 5 secs in a microfuge centrifuge and supernatants removed. Cell viability will not affected by exposure to acid. To the cell pellet, 0.5 ml of ice-cold 10 mM Tris/HCl (pH 7.4) will be added and sonicated for 10-20 secs and solubilized with 1% Triton X-100 overnight. Cells will then pelleted by centrifugation. Proteins in the supernatants of the extracts will be measured by the Bradford method (Bio-Rad). The aliquot of the solubilized extract from each sample containing an equal amount of total protein will be passed through a membrane filter with a pore size 1,000-2,000 to separate analogs from larger proteins. The filtered extract containing analogs will be chromatographed by HPLC. Another aliquot will be used for quantifying TF by ELISA. The presence of a single peak of the analog separated from the FFR-ck-VIIa, TF, FFR-ck-VIIa-TF, or analog-FFR-ck-VIIa-TF peaks will be taken as evidence of dissociation.

FFR-ck-VIIa as a negative control and the analog alone as a positive control will be added to the monolayers, cultured for 6 hours and the solubilized fraction will be similarly analyzed. HPLC will be performed using a Beckman liquid chromatograph equipped with a pump, a UV/vis. detector and a recorder. A Waters Nova-Pak C₁₈ column (150.times.3.9 mm, 5-.mu.m particle size) will be used. The mobile phase will consist of 40% tetrahydrofuran and 60% water containing 1% citric acid, adjusted to pH 3.0 with concentrated KOH solution (v/v). The system will be run isocratically at a flow rate of 1 ml/min. Sample detection will be achieved at 420 nm, and injection volumes will be 20 μl. Calibration curves over the range of 0.2 to 20 μM will be established for the quantitation of curcumin analogs. This HPLC detection method offers a detection limit of 5 ng/ml.

### Example 16

### Functional Analysis by TF and VEGF Production and Neutral Red (NR) Viability Assay

TF and VEGF levels were quantified by ELISA in the samples obtained by experiment as described above and adjusted based on protein concentration of the samples. In addition, cancer cells were grown to confluency in 48-well plates in duplicate. Each well was incubated with analog-FFR-ck-VIIa, analog alone, FFR-ck-VIIa alone, or DMSO (solvent control) for 4 days. Supernatants were collected for qualifying VEGF levels by ELISA. One plate was used to determine cell viability by NR assay. The other plate was used to determine levels of TF (in the cells) by ELISA. Levels of VEGF and TF in each well were adjusted by the value of neutral red assay.

### Example 17

### Curcuminoid EF24 is More Effective than Curcumin Against Tumor Cells

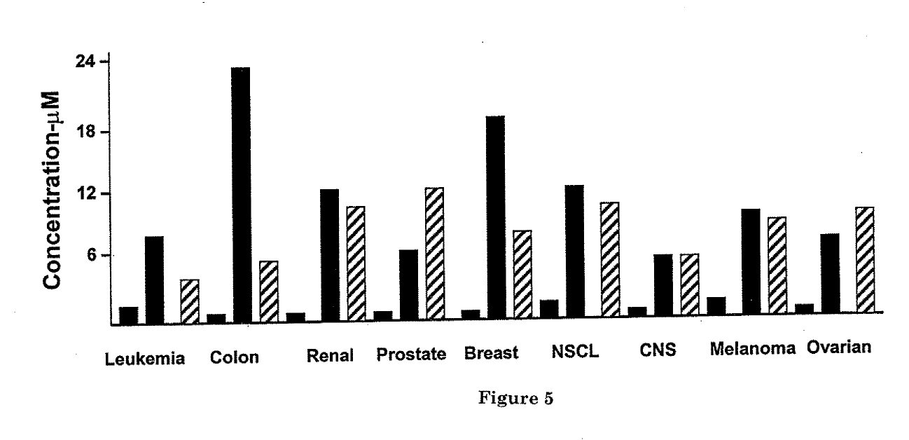

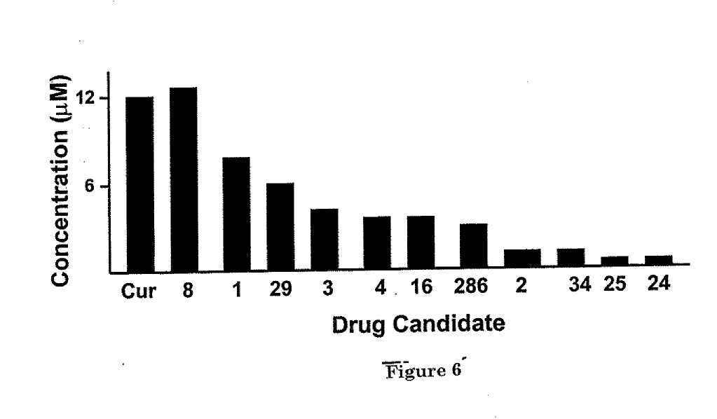

Curcumin, EF24 and cisplatin were tested against tumor cells in the NCI screening system. EF24 was significantly more effective than either cisplatin or curcumin, as shown in FIG. 5. Curcuminoids were also added to transformed breast cancer cells and the mean growth inhibitory concentrations determined, as shown in FIG. 6.

### Example 18

### Creation of Luciferase-Positive MDA-MB-231 Tumor Xenografts

MDA-MB-231 cells were co-transfected with a luciferase plasmid vector pGL2-control (Promega, Madison, Wis.) and a drug selection plasmid vector pcDNA3.1 (GIBCO/Invitrogen, Carlsbad, Calif.) at a 10:1 ratio using Lipofectamine 2000 (Invitrogen) according to the manufacturer's instruction. G418-resistant clones were single-cell cloned, and several single cell clones with the highest luciferase activity were combined and cultured together. Luciferase-positive MDA-MB-231 cells were lifted using a cell lifter (Corning, San Diego, Calif.), resuspended in Hank's BSS (GIBCO), and 2×106 cells/0.1 ml mixed with 20 μl of Matrigel/site were injected subcutaneously into flanks of female athymic nude mice (6 weeks of age from Harlan, Indianapolis, Ind.) under ketamine/xylazine anesthesia.

### Example 19

### Treatment of Breast Cancer Xenografts in Mice with the EF24-FFRck-fVIIa Conjugate

Ten days after inoculation of tumor cells, EF24 (50 μM), or the EF24-FFRck-fVIIa conjugate (containing 50 μM of EF24), or vehicle were administered intravenously through the lateral tail veins using a 27 gauge needle 2-3 times/week for a total of 5 injections five times over a span of two weeks. Eight mice per treatment regimen were used. Nineteen days following the last treatment, the luciferase activity in tumors was imaged using a Xenogen machine, and tumor sizes were measured using a caliper. No animals showed bleeding complications during the course of treatment. All protocols for animal studies were reviewed and approved by the Institutional Animal Care and Use Committee at Emory University.

To perform bioluminescence imaging of tumors, animals were anesthetized by intraperitoneal injection of a mixture of ketamine (85 mg/kg)/xylazine (10 mg/kg). The animals were then injected with luciferin (143 mg/kg, Xenogen, Alameda, Calif.) intraperitoneally, and the luciferase activity was imaged by the IVIS Imaging System (Xenogen).

Tumor blood vessels resulting from tumor angiogenesis and blood vessels recruited into Matrigels by VEGF-A in mice are genetically the same, since both are the same inbred female athymic nu/nu mice. To determine the efficacy of the EF24-FFRck-fVIIa conjugate on inhibiting tumor growth, we administered the conjugate to female nude mice bearing tumors of human breast cancer MDA-MB-231. These breast cancer cells produce high levels of both TF and VEGF. We reasoned that the EF24-FFRck-fVIIa conjugate is able to seek out and destroy both the TF-expressing tumor cells and the VECs of tumor-associated blood vessels, and thus inhibits tumor growth. To test this hypothesis, we used an animal model with subcutaneous tumors of luciferase-positive MDA-MB-231 cells. Ten days after tumor cell implantation, when the tumor size reached approximately 3-5 mm in diameter, treatments were initiated by intravenously injecting either the EF24-FFRck-fVIIa conjugate, free EF24, or control vehicle. A total of five i.v. injections, were administered over a span of 15 days. Nineteen days after the last injections, tumors on mice were imaged using a Xenogen Bioluminescence Small Animal Imager. Levels of the luciferase activity in the tumor cells are indicated by a color bar, red being the most active and blue being the least active. The luciferase activity in control animals exhibited intense color (red and yellow), which indicates high viable tumor activity. The images of mice tumors treated with the EF24-FFRck-fVIIa conjugate and free EF24 showed a blue color, indicating reduced luciferase activity due to the decreased number of viable tumor cells as compared to tumors in vehicle-treated mice. Furthermore, sizes of tumors were not apparently reduced in mice treated with free EF24. In strong contrast, significant reduction in tumor size was observed in mice treated with the EF24-FFRck-fVIIa conjugate, most likely due to the inhibition of blood vessels feeding the tumors (P<0.01: n 6). Such reduction in tumor size was evidenced by size-reduced images of the associated bioluminescence and by tumor volumes.

### Example 20

### Assay for Apoptosis in Tumor

The cleaved caspase-3 (Asp175) immunohistochemistry (IHC) Detection Kit was purchased from Cell Signaling Technology (Beverly, Mass.). All apoptotic events converged to the activation of caspase-3 that requires proteolytic cleavage of the inactive caspase-3 into activated p17 and p12 fragments. The cleaved caspase-3 antibody recognizes the p17 fragment, which results from cleavage adjacent to Asp 175, but does not recognize inactive caspase-3. Tumor xenografts were stained for apoptotic cells according to the manufacturer's instruction.

To verify the inhibitory effect of the conjugate on tumors, tumor sections from control and treated mice were stained for apoptosis by the method of cleaved caspase-3. Significant numbers of apoptotic cells were observed in the conjugate-treated tumors but not in the control tumors. This result, in which the EF24-FFRck-fVIIa conjugate causes apoptosis in tumor cells, is consistent with our previous in vitro testing that shows EF24 activates caspase-3 in MDA-MB-231 and DU-145 cells.

We claim:

1. A conjugate comprising: (a) a protein, wherein the protein selectively binds a surface marker of a target cell; (b) at least one linker covalently bonded to the protein; and (c) a cytotoxic compound bonded to the linker by a hydrolysable bond.

2. The conjugate according to claim 1, wherein the protein selectively binds to tissue factor on the surface of the target cell.

3. The conjugate according to claim 1, wherein the protein is a component polypeptide of a factor VIIa.

4. The conjugate according to claim 1, wherein the protein is a component polypeptide of a factor VIIa, or a truncated or modified variant thereof

5. The conjugate according to claim 1, wherein the protein is capable of being internalized by the target cell.

6. The conjugate according to claim 1, wherein the at least one linker is a peptidyl linker.

7. The conjugate according to claim 6, wherein the at least one peptidyl linker is a peptidyl methylketone linker.

8. The conjugate according to claim 1, wherein the composition further comprises a tether.

9. The conjugate according to claim 1, wherein the at least one linker is a tether.

10. The conjugate according to claim 1, wherein the hydrolysable bond is selected from the group consisting of a carbamate, an amide, an ester, a carbonate and a sulfonate.

11. The conjugate according to claim 1, wherein the at least one linker is an arginyl methylketone selected from the group consisting of phenylalanine-phenylalanine-arginine methylketone, tyrosine-glycine-arginine methylketone, glutamine-glycine-arginine methylketone, glutamate-glycine-arginine methylketone and phenylalanine-proline-arginine methylketone.

12. The conjugate according to claim 1, wherein the at least one linker is selected from tyrosine-glycine-arginine methylketone and phenylalanine-phenylalanine-arginine methylketone.

13. The conjugate according to claim 1, wherein the at least one linker is phenylalanine-phenylalanine-arginine methylketone.

14. The conjugate according to claim 1, wherein the at least one linker is tyrosine-glycine-arginine methylketone.

15. The conjugate according to claim 3, wherein at least one linker is covalently bonded to an amino acid side chain within a serine protease active site of factor VIIa, thereby inactivating the serine protease active site.

16. The conjugate according to claim 1, wherein the cytotoxic compound is a curcuminoid having the formula: 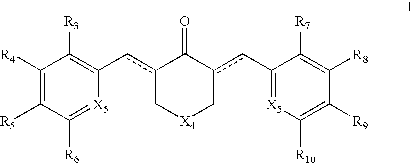
wherein: X₄ is (CH₄)ₘ, O, S, SO, SO₂, CHNH₂, CHOH, CO, or NR₁₂, where R₁₂ is H, alkyl, substituted alkyl, acyl, alkoxycarbonyl, aminocarbonyl, alkylaminocarbonyl or dialkylaminocarbonyl; m is 1-7; each X₅ is independently N or C—R₁₁; and each R₃-R₁₁ are independently H, halogen, hydroxyl, alkoxy, CF₃, alkyl, substituted alkyl, alkenyl, alkynyl, cycloalkyl, substituted cycloalkyl, aryl, substituted aryl, alkaryl, arylalkyl, heteroaryl, substituted heteroaryl, heterocycle, substituted heterocycle, amino, alkylamino, dialkylamino, carboxylic acid, carboxylic ester, carboxamide, nitro, cyano, azide. alkylcarbonyl, acyl, or trialkylammonium; and the dashed lines indicate optional double bonds; with the proviso that when X₄ is (CH₂ₘ, m is 2-6, and each X₅ is C—R₁₁, R₃-R₁₁ are not alkoxy, and when X₄ is NR₁₂ and each X₅ is N, R₃-R₁₀ are not alkoxy, alkyl, substituted alkyl, alkenyl, alkynyl, cycloalkyl, substituted cycloalkyl, aryl, substituted aryl, alkaryl, arylalkyl, heteroaryl, substituted heteroaryl, amino, alkylamino, dialkylamino, carboxylic acid, or alkylcarbonyl, and wherein the stereoisomeric configurations include enantiomers and diastereoisomers, and geometric (cis-trans) isomers.

17. The conjugate according to claim 13, wherein X₄ is selected from the group consisting of —NH and —NR₁₂.

18. The conjugate according to claim 13, wherein R₃-R₁₀ is selected from hydroxyl and —NHR₁₂.

19. The conjugate according to claim 1, wherein the cytotoxic compound is a curcuminoid having the formula:

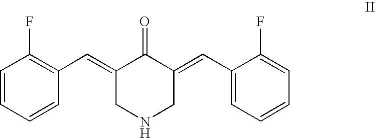

20. The conjugate according to claim 1, wherein the tether is selected from the group consisting of a dicarboxylic acid, a disulfonic acid, an omega-amino carboxylic acid, an omega-amino sulfonic acid, an omega-amino carboxysulfonic acid, or a derivative thereof, wherein the tether comprises 2-6 carbons, and wherein the tether is capable of forming a hydrolysable bond.

21. The conjugate according to claim 8, wherein the tether comprises a dicarboxylic acid.

22. The conjugate according to claim 21, wherein the tether is succinate.

23. A pharmaceutical composition comprising a conjugate comprising protein, wherein the protein selectively binds a surface marker of a target cell, and wherein the protein is covalently bonded to at least one linker, wherein each linker has a cytotoxic compound bonded thereto and the cytotoxic compound is covalently linked by hydrolysable bond to the linker, and a pharmaceutically acceptable carrier.

24. The pharmaceutical composition of claim 24 further comprising a tether covalently linked by hydrolysable bond to the cytotoxic compound.

25. The pharmaceutical composition according to claim 24, wherein the hydrolysable bond is selected from the group consisting of a carbamate, an amide, an ester, a carbonate and a sulfonate.

26. The pharmaceutical composition according to claim 24, wherein the tether is selected from the group consisting of a dicarboxylic acid, a disulfonic acid, an omega-amino carboxylic acid, an omega-amino sulfonic acid, an omega-amino carboxysulfonic acid, or a derivative thereof, wherein the tether comprises 2-6 carbons, and wherein the tether is capable of forming a hydrolysable bond.

27. The pharmaceutical composition according to claim 23, wherein the at least one linker is an arginyl methylketone selected from the group consisting of phenylalanine-phenylalanine-arginine methylketone, tyrosine-glycine-arginine methylketone, glutamine-glycine-arginine methylketone, glutamate-glycine-arginine methylketone and phenylalanine-proline-arginine methylketone.

28. The pharmaceutical composition of claim 23, wherein the cytotoxic compound is a curcuminoid having the formula

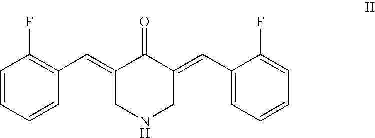

29. The pharmaceutical composition of claim 24, formulated in a pharmaceutically effective dosage amount.

30. The pharmaceutical composition of claim 23, wherein the protein is a component polypeptide of a factor VIIa.

31. The pharmaceutical composition of claim 23, wherein the pharmaceutical composition is formulated for intravenous infusion.
